(19) United States
(12) Patent Application Publication
(10) Pub. No.: US 2026/0090881 A1
(43) Pub. Date: Apr. 2, 2026

(54) PROSTHETIC FRAME COVERS

(71) Applicant: Edwards Lifesciences Corporation, Irvine, CA (US)

(72) Inventors: Radhika Vaid, Rancho Mission Viejo, CA (US); Sandip Vasant Pawar, Irvine, CA (US); Taylor Michael Winters, Stevenson Ranch, CA (US); Rupesh Gajanan Nawalakhe, Trabuco Canyon, CA (US); Aysan Rangchian, Rancho Palos Verdes, CA (US); Jessica Louise Blair, Huntington Beach, CA (US)

(21) Appl. No.: 19/411,786

(22) Filed: Dec. 8, 2025

Related U.S. Application Data
(63) Continuation of application No. PCT/US2024/032923, filed on Jun. 7, 2024.
(60) Provisional application No. 63/507,231, filed on Jun. 9, 2023.

(51) Int. Cl. A61F 2/24

(52) U.S. Cl. CPC A61F 2/2415

(57) ABSTRACT

Systems and methods for synthesis of covers for valvular prosthetics are described. Generally, covers can be synthesized via depositing, submersion, and/or printing. Covers can be synthesized directly onto a valvular frame or synthesized on a temporary substrate and transferred onto the valvular frame. Covers can be synthesized having one or more layers. The covers and the various layers can be synthesized or treated to obtain beneficial properties.

### CROSS REFERENCE TO RELATED APPLICATIONS

The current application is a continuation of International Patent Application No. PCT/US2024/032923, filed Jun. 7, 2024, which claims the benefit of U.S. patent application Ser. No. 63/507,231, filed Jun. 9, 2023, the entire disclosures all of which are incorporated by reference for all purposes.

### TECHNOLOGICAL FIELD

The disclosure is generally directed to systems and methods related to prosthetic frame covers and methods of their synthesis and attachment.

### BACKGROUND

Cardiovascular prosthetic devices are utilized to replace or assist the various parts and functions of the heart and vascular system. Examples of prosthetic devices include stents, grafts, pacemakers, sensors, and replacement heart valves. These prosthetic devices can have a number of components and be composed of various biocompatible materials. For instance, a prosthetic can comprise a frame composed of a metallic or polymeric material and cover composed of polymeric material or tissue.

Transcatheter delivery methods have become common to perform cardiovascular prosthetic device installment. Generally, a prosthetic is compressed into a catheter that is entered into the body at a peripheral artery (e.g., femoral artery) and then translocated to the site of repair for implantation. When the prosthetic reaches the site of implantation, it is expanded and set within the endogenous tissue, restoring proper cardiovascular function.

One common cardiovascular prosthetic device is a replacement heart valve. Native heart valves (such as the aortic, pulmonary, tricuspid and mitral valves) serve critical functions in assuring the forward flow of an adequate supply of blood through the cardiovascular system. These heart valves can be rendered less effective by congenital, inflammatory, or infectious conditions, resulting in disorders such as regurgitation (i.e., backflow through the valve), leaflet thickening and calcification. Such conditions can eventually lead to serious cardiovascular complications or death, and may require surgical repair and/or replacement of the valve.

Valvular stenosis and regurgitation are a few of number of complications that may necessitate a heart valve replacement. Traditional replacement valves are constructed from various biocompatible metals, polymers and animal pericardium tissue. Generally, replacement heart valves comprise a tubular expandable frame, a cover on the expandable frame, and set of leaflets attached to an inner cover or the frame such that the replacement valve provides unidirectional blood flow, while mitigating perivalvular leakage.

### SUMMARY

Systems and methods are described that synthesize covers for use on valvular protheses. Covers can be synthesized directly onto a valvular prosthetic frame or synthesize independently of the frame and then applied to the frame.

Covers can be synthesized via depositing, coating, submersion, and/or printing. Covers can be customized to have beneficial properties, which can enhance in-vivo functional and performance attributes and/or delivery of the prosthetic heart valve. Properties that can be modulated include (but are not limited to) thickness, density, hydrophilicity, hydrophobicity, layering, porosity, surface topography, edge quality, push forces, mechanical stability, term biostability and biological responsiveness (i.e., interaction with host biology).

In some aspects, the techniques described herein relate to a prosthetic heart valve, including: an expandable frame having an outer wall that is covered with an outer skirt and an inner wall that is covered with an inner cover, the outer skirt and the inner cover each composed of electrospun fibers; and a set of leaflets in connection with the inner cover within the expandable frame.

In some aspects, the techniques described herein relate to a prosthetic heart valve, wherein the outer skirt and the inner cover are a single deposit of electrospun fibers, and wherein the single deposit spans and is attached to at least a portion of the outer wall of the expandable frame to form the outer skirt and the single deposit folds across an edge of the expandable frame and spans and is attached to at least a portion of the inner wall of the expandable frame to form the inner cover.

In some aspects, the techniques described herein relate to a prosthetic heart valve, wherein the single deposit folds across at least one edge of the expandable frame.

In some aspects, the techniques described herein relate to a prosthetic heart valve, wherein the outer skirt or the inner cover is composed of electrospun fibers that include at least one of the following resorbable materials: silk fibroin, chitosan, polycaprolactone (PCL), poly(lactic-co-glycolic acid) (PGLA), poly (β-hydroxybutyrate-co-β-hydroxy valerate) (PHBV), and polyhydroxy butyrate (PHB).

In some aspects, the techniques described herein relate to a prosthetic heart valve, wherein the outer skirt or the inner cover is composed of electrospun fibers that include at least one of the following non-resorbable materials: polyolefins, polyethylene terephthalate (PET), thermoplastic polyurethane (TPU), expanded polytetrafluoroethylene (ePTFE), polyvinylpyrrolidone (PVP), and siloxanes.

In some aspects, the techniques described herein relate to a prosthetic heart valve, wherein the outer skirt has greater porosity than the inner cover.

In some aspects, the techniques described herein relate to a prosthetic heart valve, wherein the outer skirt includes more layers of electrospun fibers than the inner cover.

In some aspects, the techniques described herein relate to a prosthetic heart valve, wherein the outer skirt has a greater profile than the inner cover.

In some aspects, the techniques described herein relate to a prosthetic heart valve, wherein the inner cover is a compressed cover to reduce its profile relative to the outer skirt as well relative to an uncompressed cover.

In some aspects, the techniques described herein relate to a prosthetic heart valve, wherein the inner cover includes a biocompatible material that resists pannus overgrowth.

In some aspects, the techniques described herein relate to a prosthetic heart valve, wherein the inner cover includes fluoropolymers.

In some aspects, the techniques described herein relate to a prosthetic heart valve, wherein the inner cover includes polymers that have undergone controlled chain scission.

In some aspects, the techniques described herein relate to a prosthetic heart valve, wherein the inner cover has an even surface topography.

In some aspects, the techniques described herein relate to a prosthetic heart valve, wherein the electrospun fibers of the inner cover have been coated with TPU, the TPU coat sealing pores of the electrospun fiber layer.

In some aspects, the techniques described herein relate to a prosthetic heart valve, wherein the outer skirt is coated with an endothelialization-inducing biologic.

In some aspects, the techniques described herein relate to a prosthetic heart valve, wherein the outer skirt surface includes pattern features.

In some aspects, the techniques described herein relate to a prosthetic heart valve, wherein the outer skirt includes a gauze-like material.

In some aspects, the techniques described herein relate to a prosthetic heart valve, wherein the outer skirt has been plasma treated with O2.

In some aspects, the techniques described herein relate to a prosthetic heart valve, wherein the inner cover has been plasma treated with CF4.

In some aspects, the techniques described herein relate to a prosthetic heart valve, wherein the electrospun fibers of the outer skirt or of the inner cover includes crosslinked electrospun fibers.

In some aspects, the techniques described herein relate to a method of synthesizing a cover for a prosthetic heart valve, including: fitting an expandable frame onto a mandrel, wherein the mandrel includes or is covered with a polarized material; and releasing a polymeric solution in a controlled manner in a direction toward the mandrel to generate electrospun fibers that are deposited on the expandable frame to yield an outer skirt spanning at least a portion an outer wall of the expandable frame.

In some aspects, the techniques described herein relate to a method, further including: releasing the polymeric solution in the controlled manner in the direction toward the mandrel to generate electrospun fibers that are deposited onto the mandrel adjacent to the expandable frame, wherein the electrospun fibers that are deposited on the expandable frame and the electrospun fibers that are deposited onto the mandrel adjacent to the expandable frame are deposited in a manner to yield a single deposit; removing the expandable frame and the deposited electrospun fibers from the mandrel; and folding the portion of the electrospun fibers that were deposited onto the mandrel adjacent to the expandable frame over an edge of the expandable edge to yield an inner cover spanning at least a portion of an inner wall of the expandable frame.

In some aspects, the techniques described herein relate to a method, wherein the distance between from where a polymer solution is released and collecting material, solvent concentration, polymer concentration, and the speed of the mandrel are controlled to control thickness of the electrospun fibers or porosity among the electrospun fibers.

In some aspects, the techniques described herein relate to a method, wherein the electrospun polymers are deposited such that the outer skirt has greater porosity than the inner cover.

In some aspects, the techniques described herein relate to a method, wherein the electrospun polymers are deposited such that the outer skirt has a greater profile than the inner cover.

In some aspects, the techniques described herein relate to a method, wherein viscosity of the polymer solution is controlled by a percentage of polymer in solution to prevent beading.

In some aspects, the techniques described herein relate to a method, further including: treating the inner cover with CF4 plasma.

In some aspects, the techniques described herein relate to a method, further including: treating the outer skirt with O2 plasma.

In some aspects, the techniques described herein relate to a method, further including: prior to the deposition of electrospun fibers onto the frame, treating the frame with plasma.

In some aspects, the techniques described herein relate to a method, further including: crosslinking the deposited electrospun fibers.

In some aspects, the techniques described herein relate to a method, further including: performing a heat treatment on the deposited electrospun fibers.

In some aspects, the techniques described herein relate to a method, further including: performing a freeze-and-thaw treatment on the deposited electrospun fibers.

In some aspects, the techniques described herein relate to a method, further including: adhering a porous textile layer onto the outer skirt.

In some aspects, the techniques described herein relate to a method, further including: coating the inner cover with TPU.

In some aspects, the techniques described herein relate to a method, wherein the TPU coating seals pores of the inner cover.

In some aspects, the techniques described herein relate to a method, further including: smoothening the inner cover.

In some aspects, the techniques described herein relate to a method, further including: texturizing, embossing, or micropatterning the outer skirt.

In some aspects, the techniques described herein relate to a method to 37, further including: coating the outer skirt with an endothelialization-inducing biologic.

In some aspects, the techniques described herein relate to a method, wherein the electrospun fibers include at least one of the following resorbable materials: silk fibroin, chitosan, polycaprolactone (PCL), poly(lactic-co-glycolic acid) (PGLA), poly (β-hydroxybutyrate-co-β-hydroxy valerate) (PHBV), and polyhydroxy butyrate (PHB).

In some aspects, the techniques described herein relate to a method, wherein the electrospun fibers that include at least one of the following non-resorbable materials: polyolefins, polyethylene terephthalate (PET), thermoplastic polyurethane (TPU), expanded polytetrafluoroethylene (ePTFE), polyvinylpyrrolidone (PVP), and siloxanes.

In some aspects, the techniques described herein relate to a prosthetic for delivery via a minimally invasive procedure, including: a frame and one or more low-profile covers attached to the frame, wherein each low-profile cover includes a textile and a polymeric layer, wherein the textile includes multifilament yarns and is attached to the frame via the polymeric layer without sutures.

In some aspects, the techniques described herein relate to a prosthetic, wherein the frame is for use as: a catheter, a heart valve replacement implant, a heart valve repair implant, a vascular occlusion device, a vascular stent, or a vascular graft.

In some aspects, the techniques described herein relate to a prosthetic, wherein the textile of at least one low-profile cover is a woven textile having 60 to 100 ends per inch and 60 to 100 picks per inch,.

In some aspects, the techniques described herein relate to a prosthetic, wherein the textile of at least one low-profile cover is a woven textile having 30 to 60 ends per inch and 30 to 60 picks per inch.

In some aspects, the techniques described herein relate to a prosthetic, wherein the woven textile includes a plain weave, a twill weave, a satin weave, a leno weave, a derivatives of one of the listed weaves, or a combination of the listed weaves.

In some aspects, the techniques described herein relate to a prosthetic, wherein the textile of at least one low-profile cover is a knitted textile having 10 to 50 courses per inch and 20 to 60 wales per inch.

In some aspects, the techniques described herein relate to a prosthetic, wherein the textile of at least one low-profile cover is a braided textile having 30 to 120 picks per inch.

In some aspects, the techniques described herein relate to a prosthetic to 47, wherein the polymeric layer of at least one low-profile cover includes a material selected from: polyolefins, polyethylene terephthalate (PET), thermoplastic polyurethane (TPU), expanded polytetrafluoroethylene (ePTFE), siloxanes, and acrylics.

In some aspects, the techniques described herein relate to a prosthetic, wherein the polymeric layer includes a polymer combining thermoplastic polyurethane and polysiloxane.

In some aspects, the techniques described herein relate to a prosthetic, wherein the at least one low-profile cover has a profile less than 80 μm thick.

In some aspects, the techniques described herein relate to a prosthetic, wherein the at least one low-profile cover has a profile less than 40 μm thick.

In some aspects, the techniques described herein relate to a prosthetic, wherein the polymeric layer is a laminated or an ultrasonic coated layer.

In some aspects, the techniques described herein relate to a prosthetic to 54, wherein the multifilament yarns of at least one low-profile cover are composed of: PET, UHMWPE, PTFE, TPU, or a combination thereof.

In some aspects, the techniques described herein relate to a prosthetic to 54, the multifilament yarns of at least one low-profile cover are composed of high tenacity PET having a density of greater than 6 g/denier.

In some aspects, the techniques described herein relate to a prosthetic to 57, wherein the multifilament yarns of at least one low-profile cover are positioned about 45-degrees relative a longitudinal axis and/or a latitudinal axis of the prosthetic.

In some aspects, the techniques described herein relate to a prosthetic to 58, wherein an edge of at least one low-profile cover extends beyond a structural feature to yield an overhang.

In some aspects, the techniques described herein relate to a prosthetic, wherein a length of the overhang is between about 0.1 mm and about 1.0 mm.

In some aspects, the techniques described herein relate to a prosthetic, wherein the prosthetic is tubular, wherein a width of the overhang encircles the prosthetic.

In some aspects, the techniques described herein relate to a prosthetic, wherein the prosthetic is tubular, wherein at least one low-profile cover is an outer cover or an inner cover.

In some aspects, the techniques described herein relate to a prosthetic, wherein the one or more low-profile covers includes at least two low-profile covers, a first low-profile cover is an outer cover and second low-profile cover is an inner cover.

In some aspects, the techniques described herein relate to a prosthetic, wherein a profile thickness of the outer cover is lesser than a profile thickness of the inner cover, or a profile thickness of the inner cover is lesser than a profile thickness of the outer cover.

In some aspects, the techniques described herein relate to a prosthetic, wherein the textile of outer cover and the textile of the inner cover are each woven, wherein the textile of outer cover or the textile of the inner cover is about 30 PPI to about 60 PPI and about 30 EPI to about 60 EPI, and the textile of outer cover or the textile of the inner cover is about 60 PPI to about 100 PPI and about 60 EPI to about 100 EPI.

In some aspects, the techniques described herein relate to a prosthetic, wherein the textile of outer cover and the textile of the inner cover are the same knitted textile, wherein the knitted textile is folded over one end of the tubular prosthetic.

In some aspects, the techniques described herein relate to a prosthetic, wherein the textile of outer cover and the textile of the inner cover are each knitted, wherein the textile of outer cover and the textile of the inner cover are each about 10 CPI to about 50 CPI and 20 WPI to about 60 WPI.

In some aspects, the techniques described herein relate to a prosthetic, wherein the textile of outer cover and the textile of the inner cover are the same braided textile, wherein the braided textile is folded over one end of the tubular prosthetic.

In some aspects, the techniques described herein relate to a prosthetic to 66, wherein the inner cover has an even surface topography.

In some aspects, the techniques described herein relate to a prosthetic to 67, wherein the outer cover includes pattern features.

In some aspects, the techniques described herein relate to a prosthetic to 68, wherein the outer cover includes a coating of an endothelialization-inducing biologic.

In some aspects, the techniques described herein relate to a prosthetic to 69, wherein the prosthetic is a heart valve replacement implant.

In some aspects, the techniques described herein relate to a method to attach a low-profile cover on a prosthetic device, including: fabricating a low-profile textile; applying a tie layer to the prosthetic device; and attaching the low-profile textile to the prosthetic device onto the tie layer.

In some aspects, the techniques described herein relate to a method, wherein the step of fabricating a low-profile textile includes weft knitting or warp knitting the low-profile textile to have a density of 10 to 50 courses per inch or 20 to 60 wales per inch.

In some aspects, the techniques described herein relate to a method, wherein the step of fabricating a low-profile textile includes braiding the low-profile textile to have a density of 10 to 120 ends per inch.

In some aspects, the techniques described herein relate to a method, wherein the step of fabricating a low-profile textile includes weaving the low-profile textile to have a density of 60 to 100 ends per inch and 60 to 100 picks per inch.

In some aspects, the techniques described herein relate to a method, wherein the step of fabricating a low-profile textile includes weaving the low-profile textile to have a density of 30 to 60 ends per inch and 30 to 60 picks per inch.

In some aspects, the techniques described herein relate to a method, wherein a loom is used for weaving the low-profile textile, and wherein the ends per inch density of the low-profile textile is equal to the reed dents per inch of the loom.

In some aspects, the techniques described herein relate to a method, wherein the low-profile textile includes multifilament yarns having 2 to 160 filaments and are between 5 and 40 denier.

In some aspects, the techniques described herein relate to a method, wherein the multifilament yarns are composed of: PET, UHMWPE, PTFE, TPU, or a combination thereof.

In some aspects, the techniques described herein relate to a method, wherein the multifilament yarn is composed of high tenacity PET having a density of greater than 6 g/denier.

In some aspects, the techniques described herein relate to a method, wherein the step of applying a tie layer includes spray coating, dip coating, and laminating a material onto the prosthetic device, wherein the material is selected from: a polyolefin, polyethylene terephthalate (PET), thermoplastic polyurethane (TPU), fluorinated ethylene propylene (FEP), a siloxane, or an acrylic.

In some aspects, the techniques described herein relate to a method, wherein the prosthetic is tubular.

In some aspects, the techniques described herein relate to a method, wherein the step of applying a tie layer includes applying a tie layer to an inner surface and to an outer surface of the prosthetic.

In some aspects, the techniques described herein relate to a method, wherein the step of attaching a low-profile textile to the frame includes attaching one or more low-profile textiles to yield an inner cover on the inner surface of the prosthetic and an outer cover on the outer surface of the prosthetic.

In some aspects, the techniques described herein relate to the method, wherein the step of attaching a low-profile textile to the frame includes attaching a single low-profile textile to the frame to yield the inner cover and the outer cover, wherein the step of attaching the low-profile textile to the prosthetic further includes folding the textile over one end of the tubular prosthetic.

In some aspects, the techniques described herein relate to a method, wherein the step of attaching the low-profile textile to the prosthetic further includes orienting the frame such that the textile yarn is of about 45-degrees (±15-degrees), or about −45-degrees (±15-degrees), relative to the tubular axis.

In some aspects, the techniques described herein relate to a method to 83, wherein the step of attaching the low-profile textile to the prosthetic further includes laminating or spray coating a polymer layer onto the low-profile fabric while positioned on the prosthetic.

In some aspects, the techniques described herein relate to a method to 84 further including: compressing the low-profile textile on the frame.

In some aspects, the techniques described herein relate to a method to 85 further including: trimming off excess material of the low-profile textile.

In some aspects, the techniques described herein relate to a method, wherein the step of trimming off excess material of the low-profile textile results in the textile including an overhang that extends beyond a structural feature of the prosthetic.

In some aspects, the techniques described herein relate to a method, wherein the overhang has length between about 0.1 mm and about 1.0 mm.

In some aspects, the techniques described herein relate to a method, wherein the prosthetic is tubular and the overhang has a width that encircles the prosthetic.

In some aspects, the techniques described herein relate to a method to 89 further including: smoothening a surface of the low-profile textile.

In some aspects, the techniques described herein relate to a method to 90 further including: texturizing, embossing or micropatterning a surface of the textile.

In some aspects, the techniques described herein relate to a method to 91 further including: coating a surface of the low-profile textile with biological materials.

In some aspects, the techniques described herein relate to a method to 92, wherein the prosthetic is a heart valve replacement implant.

In some aspects, the techniques described herein relate to a method further including: attaching a set of leaflets to the prosthetic.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIG. 1A provides a schematic for depositing electrospun fibers to generate a cover.

FIG. 1B provides a schematic for ink jet printing to generate a cover.

FIG. 1C provides a schematic for ultrasonic spraying to generate a cover.

FIG. 1D provides a schematic for dipcoating to generate a cover.

FIG. 1E provides a schematic for laminating to generate a cover.

FIG. 2 provides a schematic of an apparatus for electrospinning fibers using a core solution and a sheath solution.

FIG. 3 provides a schematic of treating electrospun fibers with plasma.

FIG. 4 provides a schematic depicting examples of synthesizing a cover having multiple layers with particular functionality.

FIG. 5 provides an example of a method for fabricating and flattening a textile.

FIG. 6 provides a schematic of a multifilament yarn.

FIGS. 7A and 7B provide schematics of a plain waive and a leno weave for yielding a textile.

FIG. 7C provides an example of a woven leno weave.

FIGS. 8A and 8B provide schematics of flattening a textile via continuous roll calendaring.

FIGS. 9A, 9B, and 9C provide schematics of flattening a textile via heat press.

FIG. 10 provides an example of a prosthetic heart valve.

FIGS. 11A, 11B, 12A, and 12B provide examples of surface modifications upon a cover to promote endothelialization.

FIG. 13A provides an example of a method for attaching a textile to a frame.

FIGS. 13B and 13C provide an example of tubular frame with an attached cover having an overhang.

FIG. 14 provides results of electrospinning silk fibroin onto a substrate at various polymer substrates: 5% (panel A), 6% (panel B), 7% (panel C), 8% (panel D), 9% (panel E), and 10% (panel F). SEM images at 2500×.

FIGS. 15A and 15B provide an example of a cover.

FIGS. 16A, 16B, and 16C provide data comparing two woven fabrics with different densities.

### DETAILED DESCRIPTION

The current disclosure details systems and methods related to prosthetic implants and more specifically to covers of prosthetic implants. Although the term “cover” is utilized throughout, it is to be understood that a cover can refer to a valvular skirt or any other covering of a prosthetic implant, especially an implant having a frame or other structural component. When utilized on a tubular prosthetic, the cover can be utilized on the internal wall (also referred to as the internal surface, luminal wall, or luminal surface) or the external wall (also referred to as the external surface) of the tubular prosthetic. Further, in various instances, the internal wall or external wall of a valvular frame can be entirely or partially covered by a cover.

A cover can be utilized on any prosthetic implant that would benefit from such covering. Prosthetic implants that can utilize a cover include (but are not limited to) catheters, heart valve replacement and repair implants, vascular °Cclusion devices, vascular stents, vascular sensors, and vascular grafts. In several instances, a cover is utilized on a valvular prosthetic, such as one utilized to replace, repair, and/or support one of the four heart valves: the aortic valve, the pulmonary valve, the mitral valve, and the tricuspid valve. A cover on a prosthetic valve can provide numerous benefits. For instance, a cover can ensure that blood flows through the prosthetic valve and its leaflets. A cover can help prevent exposure of a frame or other structural component, or a portion thereof to prevent contact and/or injury to the recipient. A cover can also help integrate a prosthetic valve within the l°Cal environment that it is implanted within, allowing ingrowth of host tissue. A cover can also be utilized to mitigate paravalvular leakage.

The current disclosure is generally related to covers having desirable properties that are not provided by traditional covers. These properties are yielded via non-traditional methods for synthesizing and/or fabricating covers. Some desirable properties are covers having low profile, controlled porosity, controlled hydrophilicity/hydrophobicity, functional layering, high push forces, superior edge quality, texturing, and biological responsiveness (e.g., interaction with the host). These properties can be generated by producing covers utilizing fine denier multifilament yarns and/or utilizing a process involving additive mode of synthesis, such as (for example) depositing, coating, submersion, and/or printing. Accordingly, various processes to yield covers can include (but are not limited to) one or more of the following: weaving, knitting and/or braiding fine denier multifilament yarns, flattening processes, electrospinning, three-dimensional printing, spray coating, dip coating, and lamination. In some instances, covers are synthesized directly onto a prosthetic frame, which can yield covers that do not require sutures or adhesives for attachment. In some instances, covers are synthesized independent of the prosthetic frame and then attached to the frame; attachment can be any practical means, such as (for example) sutures, adhesives, lamination, spray coating, or a combination thereof. In some instances, covers can be partially synthesized on a substrate and then attached the frame and partially synthesized directly onto the cover attached to the frame. In some instances, covers can be partially synthesized directly onto the frame; partially synthesized on a substrate and then attached onto the partially synthesize cover on the frame. In some instances, a cover is partially synthesized directly onto one portion the frame (e.g., luminal surface) and partially synthesized on a substrate and then attached onto another part the frame (e.g., outer surface). In some instances, a woven, knitted, and/or braided cover can be further layered via a direct synthesis method. In some instances, a woven, knitted, and/or braided cover is attached to a frame and then further layered via an additive method. In one example, a frame can be mounted onto a mandrel and the external wall can be directly deposited onto the frame to form an external cover and an inner cover can be deposited onto a substrate and then attached (e.g., laminated) to the luminal wall of the frame.

Covers of the disclosure can comprise one or more layers with unique properties to provide certain functionality. For instance, selection of materials for each layer can improve the long-term stability. Furthermore, various processes can be performed on a cover layer to yield these beneficial properties. In some instances, surface chemistry modification is performed on a synthesized cover layer, such as (for example) coating with a substrate to yield a beneficial property or chemistry through material selection, solvent selection or finishing.

Various materials can be utilized to synthesize covers. Generally, any biocompatible material capable of being utilized in an additive synthesis process. Accordingly, covers can be composed of materials that can be utilized within a process comprising one or more of: electrospinning, three-dimensional printing, spray coating (including ultrasonic spray coating), dip coating, lamination, weaving, knitting, and/or braiding. Materials that can be utilized in these processes include (but are not limited to) polyolefins, polyethylene terephthalate (PET), thermoplastic polyurethane (TPU), expanded polytetrafluoroethylene (ePTFE), siloxanes, and acrylics. In some instances, polymers are combined and/or blended. For instance, polymers and various siloxanes can be blended or various copolymers can be used. For example, a copolymer of polycarbonate TPU with siloxane, a blend of silica particles with TPU or fluorinated TPUs and PETs can be utilized as materials to synthesize covers.

The described methods, systems, and devices should not be construed as limiting in any way. Instead, the present disclosure is directed toward all novel and nonobvious features and aspects of the various disclosed processes and systems, alone and in various combinations and sub-combinations with one another. The disclosed methods, systems, and devices are not limited to any specific aspect, feature, or combination thereof, nor do the disclosed methods, systems, and apparatus require that any one or more specific advantages be present or problems be solved.

Various examples of covers for prosthetics are disclosed herein, and any combination of options or properties can be utilized unless specifically excluded. For example, any of the cover layer disclosed having a particular property, can be combined with another cover layer having a particular property, even if a specific combination is not explicitly described. Likewise, the various processes for synthesizing cover layers can be mixed and matched, such as by combining any weaving process, any flattening process, any additive synthesis process, any surface modification process, etc., even if not explicitly disclosed. In short, the various covers, cover layers, functional properties, and modes of synthesis disclosed herein can be combined unless mutually exclusive or physically impossible.

Although the operations of some of the disclosed methods and processes are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

Various systems and methods utilize the synthesized covers within prosthetic devices and systems. In many instances, a prosthetic device for the purpose of implanting into a recipient. Recipients include (but are not limited to) patients, animal models, cadavers, or anthropomorphic phantoms. Accordingly, in addition to methods of treating patients, the various prosthetics can be utilized in training or other practice pr°Cedures upon animal models, cadavers, or anthropomorphic phantoms.

The described prosthetics and/or delivery systems can be sterilized, which can be performed using gamma irradiation, gas plasma, aldehydes, ethylene oxide, e-beam, dry chlorine dioxide, supercritical CO₂, X-ray and/or NiO₂. The prosthetics and/or delivery systems can be further treated with a bioburden reduction process. After preparation, the prosthetic or the delivery system can be stored within a container (in dry or wet conditions), which can be hermetically sealed or otherwise kept sterile.

### Additive Processes of Synthesizing Prosthetic Covers

Various additive processes for synthesizing valve covers can be utilized. In some instances, one or more layers of a valve cover is synthesized directly onto the prosthetic, which can be directly on a frame or on another cover layer. In some instances, one or more layers of a prosthetic cover are synthesized on a substrate and then attached to a frame. In some instances, the process of synthesizing prosthetic covers includes synthesizing multiple layers, which allows for a cover to have various properties specific to each layer. In some instances, the process includes surface modification of one or more layers, which can further provide and/or enhance particular properties of a cover (or specific layers of a cover).

Layers of prosthetic covers can be synthesized by a variety of additive processes, including (but not limited to) depositing, submersion, and/or printing. Some specific methodologies that can be employed include (but are not limited to) electrospinning, inkjet printing, spray coating, dip coating, and lamination. Utilization of one or more of these methodologies allows for a continuous or semi-continuous process for synthesis. In addition, when used to apply directly to the prosthetic frame, sutures may not be needed for attachment, reducing the steps and time for cover synthesis and attachment.

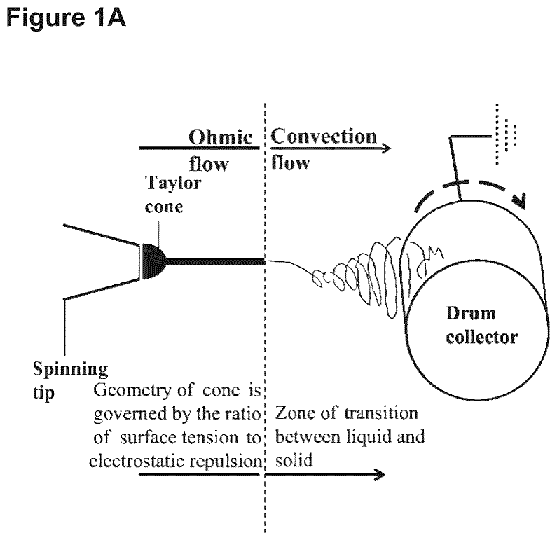

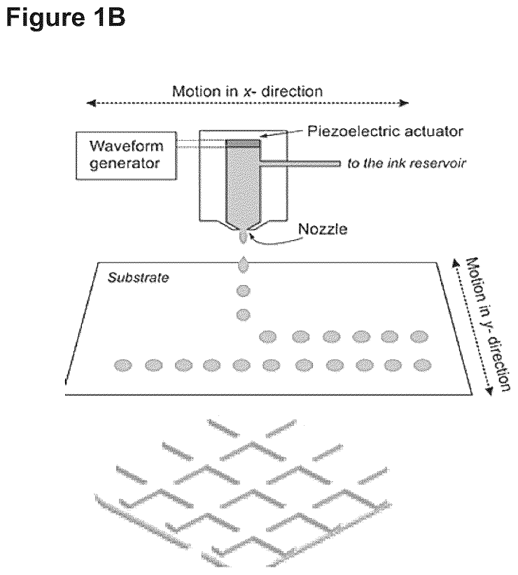

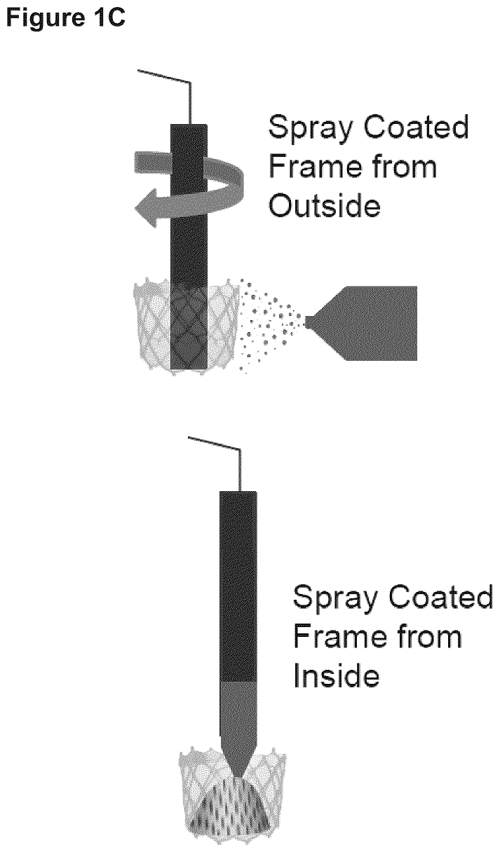

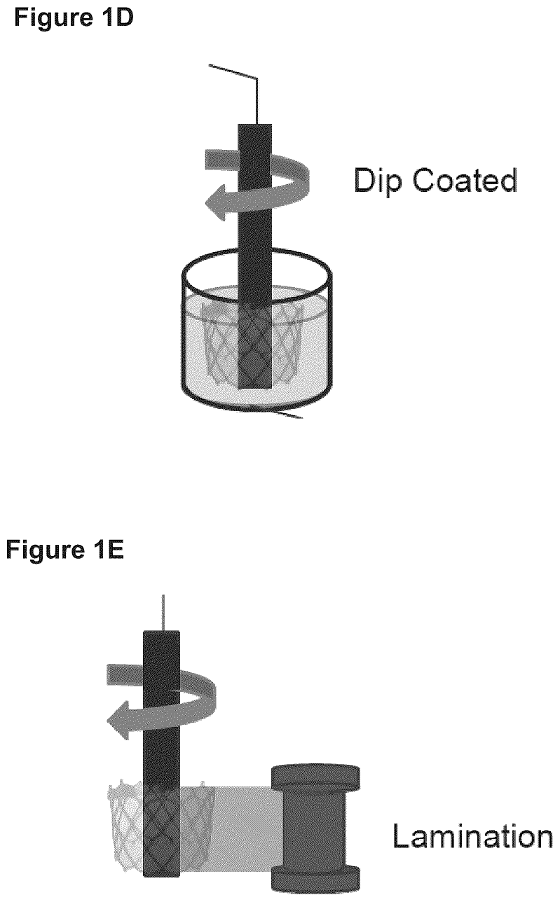

Electrospinning is a process that utilizes electric force to produce fibers or threads of polymers having a diameter under a micrometer. Generally, high voltage is applied to a droplet of solution with a polymer and a solvent, charging the droplet and generating electrostatic repulsion. The repulsion counteracts the surface tension of the droplet, resulting in stretching of the droplet until it reaches a critical point in which the liquid erupts from the surface at the Taylor cone and jet (FIG. 1A). As the jet dries and elongates in flight, the charge migrates to the surface of the fiber, elongating and thinning the polymeric fiber, resulting in formation of the nanometer diameter-sized fibers.

To perform electrospinning to generate covers, a solvent can be utilized that adequately solubilizes the polymer. In some instances, the polymer concentration within the solvent is any percentage between about 5% to about 25%. In various implementations, the polymer concentration within the solvent is about 5%, the polymer concentration within the solvent is about 10%, the polymer concentration within the solvent is about 15%, the polymer concentration within the solvent is about 20%, or the polymer concentration within the solvent is about 25%. In regards to polymer concentration, “about” is meant to be ±2.5%. In some implementations, the material utilized is resolvable such that it breaks down over time after implementation. In some implementations, the material utilized is nonresolvable, which can provide long term (or lifetime) integration after implantation. Resolvable polymers that can be utilized include (but are not limited to) silk fibroin, chitosan, polycaprolactone (PCL), poly(lactic-co-glycolic acid) (PGLA), poly (β-hydroxybutyrate-co-β-hydroxy valerate) (PHBV), and polyhydroxy butyrate (PHB). Nonresolvable polymers that can be utilized include (but are not limited to) polyolefins, polyethylene terephthalate (PET), thermoplastic polyurethane (TPU), expanded polytetrafluoroethylene (ePTFE), polyvinylpyrrolidone (PVP), and siloxanes; which can be combined and/or fluorinated. Solvents that can be utilized include (but are not limited to) tetrahydrofuran (THF), dimethylacetamide (DMAc), hexafluoroisopropanol (HFIP), acetone, ethyl acetate, acetic acid, and trifluoroacetic acid.

In some implementations, TPU is combined with polysiloxane, which yield polymers that are biostable, biocompatible, and less immunogenic. The ratio of TPU to polysiloxane can vary. In some implementations, when TPU is combined with polysiloxane, the polysiloxane is between about 5% and about 25%. In various implementations, the percentage of polysiloxane is about 5%, the percentage of polysiloxane is about 10%, the percentage of polysiloxane is about 15%, the percentage of polysiloxane is about 20%, or the percentage of polysiloxane is about 25%. In regards to siloxane percentage, “about” is meant to be ±2.5%. Any of a variety of TPUs can be utilized. The soft segment of the TPU can be polyester, polyether, or polycaprolactone. The hard segment of TPU can be aromatic or aliphatic. The TPU can further be carbonate based (i.e., comprise carbonate linkages).

It has been found that covers synthesized with a polymer having a Shore durometer of 45 A to 100 A provide good tissue response. Accordingly, in various implementations, the Shore durometer of a polymer is about 45 A, the Shore durometer of a polymer is about 50 A, the Shore durometer of a polymer is about 55 A, the Shore durometer of a polymer is about 60 A, the Shore durometer of a polymer is about 65 A, the Shore durometer of a polymer is about 70 A, the Shore durometer of a polymer is about 75 A, the Shore durometer of a polymer is about 80 A, the Shore durometer of a polymer is about 85 A, the Shore durometer of a polymer is about 90 A, the Shore durometer of a polymer is about 95 A, the Shore durometer of a polymer is about 100 A. In regards to Shore durometer, “about” is meant to be ±2.5 A.

The high-voltage power supply can be voltage range of about 5 kV to 50 kV, including examples of values of about 10 kV, about 15 kV, about 20 kV, about 25 kV, about 30 kV, about 35 kV, about 40 kV, and about 45 kV, direct-current power supply. In regards to high voltage power supply, “about” is meant to be ±5 kV.

Electrospinning distance can be dependent on the desired properties and can range from about 0.1 cm to about 200 cm, including examples of values of 0.5 cm, about 1 cm, about 5 cm, about 10 cm, about 20 cm, about 50 cm, about 100 cm, about 125 cm, about 150 cm, and about 175 cm. It is understood that at shorter distances, larger fibers can be obtained. In regards to electrospinning distance, “about” is to mean ±10% of the length.

The rotational speed of the collecting mandrel can also be any value from greater than 0 rpm to about 1,200 rpm, including examples of values of about 5 rpm, about 10 rpm, about 20 rpm, about 50 rpm, about 100 rpm, about 200 rpm, about 300 rpm, about 400 rpm, about 500 rpm, about 600 rpm, about 700 rpm, about 800 rpm, about 900 rpm, about 1,000 rpm, and about 1,110 rpm. In regards to electrospinning speed, “about” is to mean ±10% rpm.

A nonwoven layer of electrospun fibers can be directly deposited onto a prosthetic frame. Alternatively, a textile can be integrated with the frame (e.g., via sutures, lamination, or other means) and then a polymeric cover is electrospun onto the textile. And in some instances, a nonwoven layer of electrospun fibers can be deposited onto a substrate or a textile, and then attached to the frame with or without the substrate/textile. Further, one or more additional layers of the polymer cover can be electrospun on prior electrospun polymeric coats. Temperature and pressure can be applied to ensure attachment of the polymeric cover. In some instances, the attachment temperature for electrospinning a cover is any temperature between about 60° C. and about 330° C. In various implementations, the temperature for electrospinning a cover is about 60° C., the temperature for electrospinning a cover is about 80° C., the temperature for electrospinning a cover is about 100° C., the temperature for electrospinning a cover is about 120° C., the temperature for electrospinning a cover is about 140° C., the temperature for electrospinning a cover is about 160° C., the temperature for electrospinning a cover is about 180° C., the temperature for electrospinning a cover is about 200° C., the temperature for electrospinning a cover is about 220° C., the temperature for electrospinning a cover is about 240° C., the temperature for electrospinning a cover is about 260° C., the temperature for electrospinning a cover is about 280° C., the temperature for electrospinning a cover is about 300° C., the temperature for electrospinning a cover is about 320° C., or the temperature for electrospinning a cover is about 330° C. In regards to temperature for electrospinning a cover, “about” is to mean ±10° C. In some instances, the pressure for electrospinning attachment is any pressure between 0.8 MPa and 5 MPa. In various implementations, the pressure for electrospinning attachment is about 1 MPa, the pressure for electrospinning attachment is about 2 MPa, the pressure for electrospinning attachment is about 3 MPa, the pressure for electrospinning attachment is about 4 MPa, or the pressure for electrospinning attachment is about 5 MPa. In regards to pressure for electrospinning attachment, “about” is to mean ±0.5 MPa. After electrospinning a polymer, the polymer coat can be dried, cured, and/or washed.

Inkjet printing is process in which droplets of “ink” are deposited in specific l°Cations. Use of polymers or metals as ink allows for additive manufacture of various structures, devices, and components. Generally, the polymer is provided in solvent to yield an ink solution and inkjet printing can be utilized to deposit polymers onto a substrate to yield a cover on the substrate (FIG. 1B).

To perform additive printing to generate covers, a solvent can be utilized that adequately solubilizes the polymer. In some instances, the polymer concentration within the solvent is any percentage between about 5% to about 25%. In various implementations, the polymer concentration within the solvent is about 5%, the polymer concentration within the solvent is about 10%, the polymer concentration within the solvent is about 15%, the polymer concentration within the solvent is about 20%, or the polymer concentration within the solvent is about 25%. In regards to polymer concentration, “about” is meant to be ±2.5%. Polymers that can be utilized include (but are not limited to) Polyolefins, thermoplastic polyurethane (TPU), expanded polytetrafluoroethylene (ePTFE), and siloxanes; which can be combined and/or fluorinated. Solvents that can be utilized include (but are not limited to) tetrahydrofuran (THF), dimethylacetamide (DMAc), hexafluoroisopropanol (HFIP), acetone, and ethyl acetate.

A polymeric cover can be printed directly onto a prosthetic frame. Alternatively, a textile can be integrated with the frame (e.g., via sutures or other means) and then a polymeric cover is printed onto the textile. And in some instances, a polymeric cover can be deposited onto a substrate or a textile, and then attached to the frame with or without the substrate/textile. Further, one or more additional layers of the polymer cover can be printed on prior printed polymeric coats. Temperature and pressure can be applied to ensure attachment of the polymeric cover. In some instances, the attachment temperature or treatment temperature (e.g., for thermoplastic material printed cover) or sintering temperature (e.g., for ePTFE printed component) is any temperature between about 100° C. and about 400° C. In various implementations, the temperature for printing, treating, or sintering a cover is about 100° C., the temperature for printing, treating, or sintering a cover is about 120° C., the temperature for printing, treating, or sintering a cover is about 140° C., the temperature for printing, treating, or sintering a cover is about 160° C., the temperature for printing, treating, or sintering a cover is about 180° C., the temperature for printing, treating, or sintering a cover is about 200° C., the temperature for printing, treating, or sintering a cover is about 220° C., the temperature for printing, treating, or sintering a cover is about 240° C., the temperature for printing, treating, or sintering a cover is about 260° C., the temperature for printing, treating, or sintering a cover is about 280° C., the temperature for printing, treating, or sintering a cover is about 300° C., the temperature for printing, treating, or sintering a cover is about 320° C., the temperature for printing, treating, or sintering a cover is about 340° C., the temperature for printing, treating, or sintering a cover is about 360° C., the temperature for printing, treating, or sintering a cover is about 380° C., or the temperature for printing, treating, or sintering a cover is about 400° C. In regards to temperature for printing, treating, or sintering a cover, “about” is to mean ±10° C. In some instances, the pressure for printing attachment is any pressure between about 0.1 MPa and about 5 MPa. In various implementations, the pressure for printing attachment is about 1 MPa, the pressure for printing attachment is about 2 MPa, the pressure for printing attachment is about 3 MPa, the pressure for printing attachment is about 4 MPa, or the pressure for printing attachment is about 5 MPa. In regards to pressure for printing attachment, “about” is to mean ±0.5 MPa. After printing a polymer, the polymer coat can be dried, cured, and/or washed.

Spray coating is a process in which polymers are sprayed onto a substrate. Various types of spray coating can be performed, including (but not limited to) pneumatic spray coating and ultrasonic spray coating. Pneumatic spray coating uses compressed air to deposit the polymer through a nozzle. Ultrasonic spray coating utilizes a spray nozzle that sprays a polymer via high frequency vibrations that create capillary waves of fluid. Once the amplitude of the capillary waves reaches a critical height, tiny droplets of fluid fall of the nozzle, resulting in atomization of the fluid (FIG. 1C). Several parameters can be controlled to yield particular droplet sizes, including frequency of vibration, surface tension, and viscosity of the fluid. The higher the frequency, the smaller the droplet size and generally frequencies to deposit polymers are in the range of 25 kHz to 180 kHz.

Various nozzle conformations can be utilized to control ultrasonic spray shaping. For instance, nozzles can provide fine lines and controlled sprays as thin as 0.5 mm to 25 mm. Alternatively, large spray shapes, up to 25 cm, can be generated. In addition, vortex nozzles can produce a conical spray, which can be useful for spraying onto a luminal wall of a tubular frame. Accordingly, ultrasonic spray coating can be utilized to quickly and efficiently spray a polymer cover onto prosthetic frame.

To generate covers via ultrasonic spray, a solvent can be utilized that adequately solubilizes the polymer. In some instances, the polymer concentration within the solvent is any percentage between 0.5% to 25%. In various implementations, the polymer concentration within the solvent is about 0.5%, the polymer concentration within the solvent is about 5%, the polymer concentration within the solvent is about 10%, the polymer concentration within the solvent is about 15%, the polymer concentration within the solvent is about 20%, or the polymer concentration within the solvent is about 25%. In regards to polymer concentration, “about” is meant to be ±2.5%. Polymers that can be utilized include (but are not limited to) polyolefins, thermoplastic polyurethane (TPU), expanded polytetrafluoroethylene (ePTFE), and siloxanes; which can be combined and/or fluorinated. Solvents that can be utilized include (but are not limited to) tetrahydrofuran (THF), dimethylacetamide (DMAc), hexafluoroisopropanol (HFIP), acetone, and ethyl acetate.

In some implementations, TPU is combined with polysiloxane, which yield polymers that are biostable, biocompatible, and less immunogenic. The ratio of TPU to polysiloxane can vary. In some implementations, when TPU is combined with polysiloxane, the polysiloxane is between about 5% and about 25%. In various implementations, the percentage of polysiloxane is about 5%, the percentage of polysiloxane is about 10%, the percentage of polysiloxane is about 15%, the percentage of polysiloxane is about 20%, or the percentage of polysiloxane is about 25%. In regards to siloxane percentage, “about” is meant to be ±2.5%. Any of a variety of TPUs can be utilized. The soft segment of the TPU can be polyester, polyether, or polycaprolactone. The hard segment of TPU can be aromatic or aliphatic. The TPU can further be carbonate based (i.e., comprise carbonate linkages).

It has been found that covers coated with a polymer having a Shore durometer of 45 A to 100 A provide good tissue response. Accordingly, in various implementations, the Shore durometer of a polymer is about 45 A, the Shore durometer of a polymer is about 50 A, the Shore durometer of a polymer is about 55 A, the Shore durometer of a polymer is about 60 A, the Shore durometer of a polymer is about 65 A, the Shore durometer of a polymer is about 70 A, the Shore durometer of a polymer is about 75 A, the Shore durometer of a polymer is about 80 A, the Shore durometer of a polymer is about 85 A, the Shore durometer of a polymer is about 90 A, the Shore durometer of a polymer is about 95 A, the Shore durometer of a polymer is about 100 A. In regards to Shore durometer, “about” is meant to be ±2.5 A.

A polymeric cover can be sprayed directly onto a prosthetic frame. If the frame is tubular, the frame can be placed on a mandrel or other tool to assist in coating. Alternatively, a textile can be integrated with the frame (e.g., via sutures, lamination or other means) and then a polymeric cover is sprayed onto the textile. And in some instances, a polymeric cover can be deposited onto a substrate or a textile, and then attached to the frame with or without the substrate/textile. And in some instances, one or more layers of polymeric cover are sprayed onto a prosthetic frame, a textile is integrated on top of the deposited layers (e.g., via sutures, lamination or other means). Further, one or more additional layers of the polymer cover can be sprayed on prior sprayed polymeric coats and/or integrated textiles.

The spray coating flow rate can vary. In some implementations, the coating flowrate is between about 0.5 mL/min to about 8 mL/min. In various implementations, the coating flowrate is about 1.0 mL/min, the coating flowrate is about 2.0 mL/min, the coating flowrate is about 3.0 mL/min, the coating flowrate is about 4.0 mL/min, the coating flowrate is about 5.0 mL/min, the coating flowrate is about 6.0 mL/min, the coating flowrate is about 7.0 mL/min, or the coating flowrate is about 8.0 mL/min. In regards to spray coating flowrate, “about” is meant to be ±0.5 mL/min.

Temperature and pressure can be applied to ensure attachment of the polymeric cover. In some instances, the attachment temperature for spraying a cover is any temperature between 100° C. and 400° C. In various implementations, the temperature for spraying a cover is about 100° C., the temperature for spraying a cover is about 120° C., the temperature for spraying a cover is about 140° C., the temperature for spraying a cover is about 160° C., the temperature for spraying a cover is about 160° C., the temperature for spraying a cover is about 160° C., the temperature for spraying a cover is about 160° C., the temperature for spraying a cover is about 160° C., the temperature for spraying a cover is about 160° C., the temperature for spraying a cover is about 160° C., the temperature for spraying a cover is about 160° C., the temperature for spraying a cover is about 160° C., the temperature for spraying a cover is about 160° C., the temperature for spraying a cover is about 160° C., the temperature for spraying a cover is about 160° C., or the temperature for spraying a cover is about 400° C. In regards to temperature for spraying a cover, “about” is to mean ±10° C. In some instances, the pressure for spraying attachment is any pressure between about 0.1 MPa and about 5 MPa. In various implementations, the pressure for spraying attachment is about 1 MPa, the pressure for spraying attachment is about 2 MPa, the pressure for spraying attachment is about 3 MPa, the pressure for spraying attachment is about 4 MPa, or the pressure for spraying attachment is about 5 MPa. In regards to pressure for printing attachment, “about” is to mean ±0.5 MPa. After spraying a polymer, the polymer coat can be dried, cured, and/or washed.

In some implementations, the thickness of a sprayed coat can be between about 10 μm and about 120 μm and the added weight of a sprayed coat can be between about 15 g/m² to about 30 g/m². In various implementations, the thickness of a sprayed coat is about 20 μm, the thickness of a sprayed coat is about 40 μm, the thickness of a sprayed coat is about 60 μm, the thickness of a sprayed coat is about 80 μm, the thickness of a sprayed coat is about 100 μm, or the thickness of a sprayed coat is about 120 μm. In regards to thickness of sprayed coat, “about” is meant to be ±10 μm. In various implementations, the added weight of a sprayed coat is about 15 g/m², the added weight definition of a sprayed coat is about 20 g/m², the added weight of a sprayed coat is about 25 g/m², or the added weight of a sprayed coat is about 30 g/m². In regards to added weight of sprayed coat, “about” is meant to be ±2.5 g/m².

When spraying to coat a textile material, the polymer can be provided in a solvent at a concentration between 0.5% and 8%, using any of the polymers listed above for spray coating. To coat a textile, any appropriate method can be utilized. In some instances, a textile is placed onto a plate or mandrel that has a lubricious coating. A coat of polymer can be sprayed onto the textile. If both sides of the textile is to be coated, a coat of polymer can be sprayed on top of the lubricious coating prior to placing the textile thereupon.

When spraying to coat a prosthetic frame, the frame can first be cleaned to yield an unsullied and sterile frame. Cleaning can be performed with any appropriate reagent, such as (for example) high percentage isopropyl alcohol (e.g., 91% or 99%). A prosthetic frame can further be treated with plasma and/or corona to remove any residual oil, induce functionality, and/or provide a roughness to the frame surface. A primer layer (or tie layer) of polymer can be sprayed directly onto the treated frame, utilizing the polymers and concentrations described above. Upon coating with a primer layer, a textile can be placed thereupon and/or further layers can be coated thereupon. In some implementations, the textile is precoated and/or laminated. In some implementations, the textile is placed upon the frame such that yarn orientation has an angle of about 45-degrees (±15-degrees) relative to the longitudinal axis and/or latitudinal axis of the prosthetic.

Dip coating is a process in which a substrate is submerged into a solution, resulting in a coating the substrate with a polymer coating (FIG. 1D). The thickness of the coating can be controlled by the polymer type, the length of immersion, and other reaction factors (e.g., temperature, pressure).

To perform dip coating to generate covers, a dipping solvent can be utilized that adequately solubilizes the polymer. In some instances, the polymer concentration within the solvent is any percentage between 0.5% to 25%. In various implementations, the polymer concentration within the solvent is about 0.5%, the polymer concentration within the solvent is about 5%, the polymer concentration within the solvent is about 10%, the polymer concentration within the solvent is about 15%, the polymer concentration within the solvent is about 20%, or the polymer concentration within the solvent is about 25%. In regards to polymer concentration, “about” is meant to be ±2.5%. Polymers that can be utilized include (but are not limited to) polyolefins, thermoplastic polyurethane (TPU), expanded polytetrafluoroethylene (ePTFE), and siloxanes; which can be combined and/or fluorinated. Solvents that can be utilized include (but are not limited to) tetrahydrofuran (THF), dimethylacetamide (DMAc), hexafluoroisopropanol (HFIP), acetone, and ethyl acetate.

A prosthetic frame can be immersed in the polymeric solution to generate a polymeric cover. Alternatively, a textile can be integrated with the frame (e.g., via sutures or other means) and then a polymeric cover is coated onto the textile. And in some instances, a substrate or a textile can be immersed into a polymeric solution onto, and then attached to the frame with or without the substrate/textile. Further, one or more additional layers of the polymer cover can be coated on prior dip coated polymeric coats. Temperature and pressure can be applied to ensure attachment of the polymeric cover. In various instances, the attachment temperature for dip coating a cover is any temperature between 100° C. and 400° C. In various implementations, the temperature for dip coating a cover is about 100° C., the temperature for dip coating a cover is about 120° C., the temperature for dip coating a cover is about 140° C., the temperature for dip coating a cover is about 160° C., the temperature for dip coating a cover is about 180° C., the temperature for dip coating a cover is about 200° C., the temperature for dip coating a cover is about 220° C., the temperature for dip coating a cover is about 240° C., the temperature for dip coating a cover is about 260° C., the temperature for dip coating a cover is about 280° C., the temperature for dip coating a cover is about 300° C., the temperature for dip coating a cover is about 320° C., the temperature for dip coating a cover is about 340° C., the temperature for dip coating a cover is about 360° C., the temperature for dip coating a cover is about 380° C., or the temperature for dip coating a cover is about 400° C. In regards to temperature for dip coating a cover, “about” is to mean ±10° C. In some instances, the pressure for dip coating attachment is any pressure between about 0.5 MPa and about 5 MPa. In various implementations, the pressure for dip coating attachment is about 1 MPa, the pressure for dip coating attachment is about 2 MPa, the pressure for dip coating attachment is about 3 MPa, the pressure for dip coating attachment is about 4 MPa, or the pressure for dip coating attachment is about 5 MPa. In regards to pressure for printing attachment, “about” is to mean ±0.5 MPa. After dip coating a polymer, the polymer coat can be dried, cured, and/or washed.

Lamination is the process of adhering a layer of polymeric material onto a substrate (FIG. 1E). To perform lamination, a paper, fabric, cloth or other thin absorbable material is saturated with a polymeric solution to yield a lamination film. The lamination film is then adhered to the substrate utilizing appropriate temperature and pressure settings. Typically, the film is heated and/or pressurized above its melting point and then adhered and/or molded onto a substrate.

To perform lamination to generate covers, a film can be utilized that comprises the polymer. Polymers that can be utilized include (but are not limited to) polyolefins, thermoplastic polyurethane (TPU), expanded polytetrafluoroethylene (ePTFE), treated polytetrafluoroethylene (PTFE) (e.g., treated with plasma, corona or other chemical finishing), and siloxanes; which can be combined and/or fluorinated. Solvents that can be utilized include (but are not limited to) tetrahydrofuran (THF), dimethylacetamide (DMAc), hexafluoroisopropanol (HFIP), acetone, and ethyl acetate.

In some implementations, TPU is combined with polysiloxane, which yield polymers that are biostable, biocompatible, and less immunogenic. The ratio of TPU to polysiloxane can vary. In some implementations, when TPU is combined with polysiloxane, the polysiloxane is between about 5% and about 25%. In various implementations, the percentage of polysiloxane is about 5%, the percentage of polysiloxane is about 10%, the percentage of polysiloxane is about 15%, the percentage of polysiloxane is about 20%, or the percentage of polysiloxane is about 25%. In regards to siloxane percentage, “about” is meant to be ±2.5%. Any of a variety of TPUs can be utilized. The soft segment of the TPU can be polyester, polyether, or polycaprolactone. The hard segment of TPU can be aromatic or aliphatic. The TPU can further be carbonate based (i.e., comprise carbonate linkages).

It has been found that laminates with a polymer having a Shore durometer of 45 A to 100 A provide good tissue response. Accordingly, in various implementations, the Shore durometer of a polymer is about 45 A, the Shore durometer of a polymer is about 50 A, the Shore durometer of a polymer is about 55 A, the Shore durometer of a polymer is about 60 A, the Shore durometer of a polymer is about 65 A, the Shore durometer of a polymer is about 70 A, the Shore durometer of a polymer is about 75 A, the Shore durometer of a polymer is about 80 A, the Shore durometer of a polymer is about 85A, the Shore durometer of a polymer is about 90 A, the Shore durometer of a polymer is about 95 A, the Shore durometer of a polymer is about 100 A. In regards to Shore durometer, “about” is meant to be ±2.5 A.

A prosthetic frame can be laminated by the polymeric film to generate a polymeric cover. Alternatively, a textile can be integrated with the frame (e.g., via sutures or other means) and then a polymeric cover is laminated onto the textile. And in some instances, a substrate or a textile can be laminated by the polymeric film, and then attached to the frame with or without the substrate/textile. Further, one or more additional layers of the polymer cover can be laminated on prior laminated polymeric coats. And in some instances, a textile is utilized as a laminate and is adhered to the frame via lamination.

Temperature and pressure can be applied to ensure adhesion of the polymeric cover. In some instances, the adhesion temperature for laminating a cover is any temperature between 80° C. and 250° C. In various implementations, the temperature for dip coating a cover is about 120° C., the adhesion temperature for laminating a cover is about 130° C., the adhesion temperature for laminating a cover is about 140° C., the adhesion temperature for laminating a cover is about 150° C., the adhesion temperature for laminating a cover is about 160° C., the adhesion temperature for laminating a cover is about 170° C., the adhesion temperature for laminating a cover is about 180° C., the adhesion temperature for laminating a cover is about 190° C., the adhesion temperature for laminating a cover is about 200° C., the adhesion temperature for laminating a cover is about 210° C., the adhesion temperature for laminating a cover is about 220° C., the adhesion temperature for laminating a cover is about 230° C., the adhesion temperature for laminating a cover is about 240° C., or the adhesion temperature for laminating a cover is about 250° C. In regards to adhesion temperature for laminating a cover, “about” is to mean ±5° C. In some instances, the pressure for lamination is any pressure between about 0.5 MPa and about 5 MPa. In various implementations, the pressure for d lamination is about 1 MPa, the pressure for lamination is about 2 MPa, the pressure for lamination is about 3 MPa, the pressure for lamination is about 4 MPa, the pressure for lamination is about 5 MPa. In regards to pressure for lamination, “about” is to mean ±0.5 MPa. After laminating a polymer, the polymer coat can be dried, cured, and/or washed.

Polymeric covers can be synthesized onto a textile and then processed for attachment or adhesion to the prosthetic frame. Accordingly, textiles can be generated and then TPU or other polymers can be deposited on the fabrics via the synthetic additive mechanisms described, especially via electrospinning, inkjet printing, ultrasonic spraying, or lamination. Alternatively, the synthesized materials can be synthesized onto a temporary substrate, removed from the substrate, and then shaped for attachment onto the frame. Polymeric textiles can also be generated via polymeric yarn threads that are woven, knitted, nonwoven and/or braided. A polymeric yarn thread can be monofilament or multifilament or twisted or textured or braided. Any methodology to shape a textile can be utilized, such as heat setting, ultrasonic welding, die cutting or laser cutting. In some instances, a fabric is laser cut at an angle to yield an edge of threads between 30 to 60 degrees. In some particular instances, a textile is laser cut an angle to yield an edge of threads at a 45-degree angle (or thereabout). Cutting the textile at angle can improve the fabric's ability to stretch, which can prevent tearing during crimping and expansion.

Textiles can be generated from a variety of polymeric materials, including (but not limited to) PET, UHMWPE, PTFE, TPU, and combinations thereof. Fabricated textiles can have a various densities, which is partially based on the method of synthesis. Low profile fabrics with thickness 20-40 μm, can be generated via heat (100-220 ° C.) and pressure (0.5-2 MPa) assisted compaction or compression using a heat press or calendar.

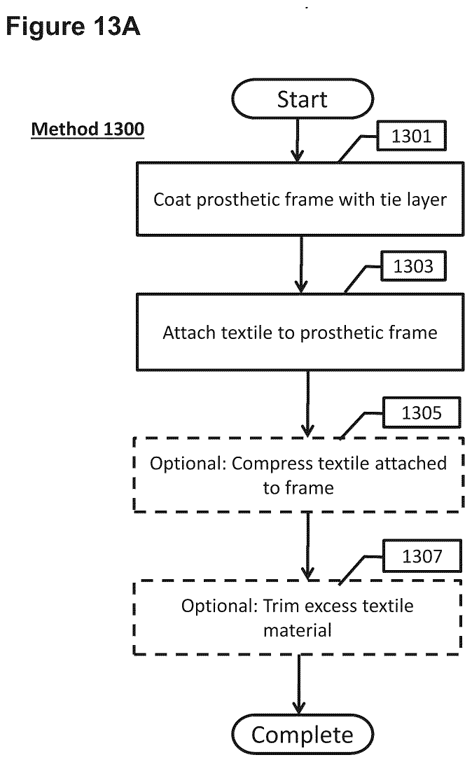

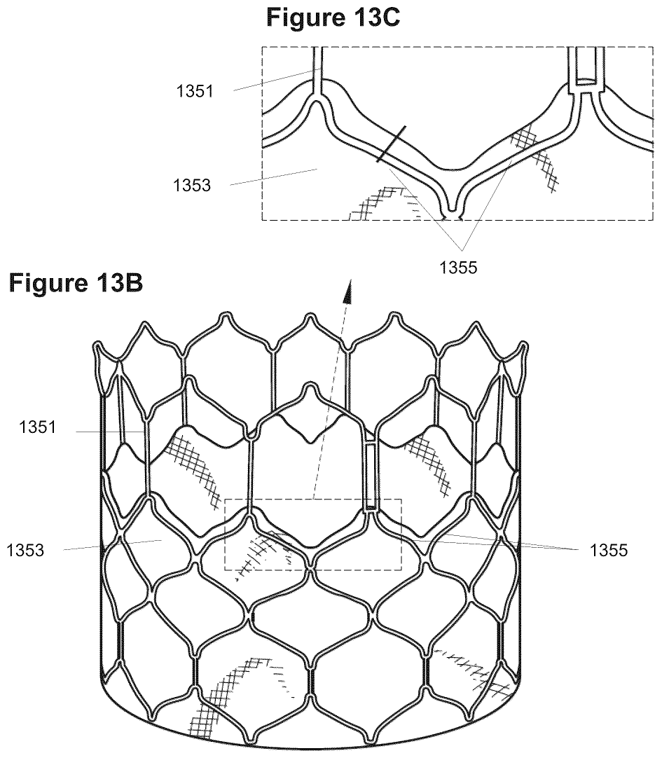

In some implementations, a textile is utilized as a laminate and laminated onto a prosthetic frame to yield a cover. A planar or tubular textile can be synthesized, as described herein. In some implementations, the textile is further coated with polymeric layer. The textile can be placed upon a prosthetic frame and then laminated there upon, using temperatures and pressures as described herein. Prior to lamination, the textile can be positioned such that yarn orientation has an angle of about 45-degrees (±15-degrees) relative to the longitudinal axis and/or latitudinal axis of the prosthetic. If the prosthetic frame is tubular, a tubular textile can be fitted on the outer surface or inner surface prior to lamination. Further, a planar textile can be fitted onto the outer surface and/or inner surface of a tubular frame and a seam can be formed to form a tubular shape by abutting or overlapping two opposite ends to form a tubular shape and using a TPU or other polymer to seal the seam. In some instances, a textile is synthesized in tubular shape, e.g., by knitting or braiding. Generally, the TPU or other polymer for lamination should have a glass transition temperature (Tg) similar to that of the polymer of the textile, or the polymer coated onto the textile. In some implementations, a textile is laminated onto an outer surface and an inner surface of a tubular frame, which can be achieved using two textiles or a single textile that is folded over an edge of the tubular frame. A cover laminated onto a frame can be trimmed via laser cutting or any other technique that yields high quality edges. Further description of laser cutting is described in reference to FIGS. 13B and 13C.

Whether covers are directly synthesized on the prosthetic frame or synthesized independently of the prosthetic frame and then attached, the cover can comprise one or more layers. When a plurality of layers is utilized, each layer can have unique properties. For instance, one or more layers may have properties for adherence to the frame. One or more layers can provide mechanical support. One or more layers may promote tissue ingrowth and endothelialization. Alternatively, one or more layers may resist tissue ingrowth and endothelialization. Certain orders of layers may be desirable. For instance, layers for adherence to a prosthetic frame can be in contact or nearest to the frame whereas layers for promoting tissue ingrowth and endothelialization are along the external surface that would be in contact with the native tissue when implanted. Layers for resisting tissue ingrowth and endothelialization can be along the surface configured to be facing towards a vascular lumen (or other non-tissue location) to prevent tissue growth within the lumen. Layers for providing mechanical reinforcement can be provided therebetween. In some implementations, a layer for promoting tissue ingrowth and endothelialization is provided as the most external layer of the prosthetic configured to be in contact with native tissue such that the layer can promote tissue regeneration. In some implementations, a layer for resisting tissue ingrowth is provided as the most luminal layer of the prosthetic as it is ideal to prevent any tissue ingrowth or pannus formation on within a vascular lumen.

Covers can be synthesized for various cardiovascular implantable devices, which can each have a unique shape or form. Some implantable devices can be in the form of a sheet, having two major faces. In some implementations, the cover of one face or both faces of the sheet are configured to be in contact with native tissue. In some implementations, the cover of one face or both faces of the sheet are configured to be facing towards a vascular lumen. Some implantable devices can be in the form of a three-dimensional object, having a plurality of faces. In some implementations, the cover of one or more faces of the three-dimensional object are configured to be in contact with native tissue. In some implementations, the cover of one or more faces of the three-dimensional object are configured to be facing towards a vascular lumen. Some implantable devices can be in the form of a tubular sheet, having an internal face and an external face. In some implementations, the cover of the external face of the tubular sheet is configured to be in contact with native tissue. In some implementations, the cover of the internal face of the tubular sheet is configured to be facing towards a vascular lumen.

Various material may be advantageous to utilize for particular layers, depending on the particular function it may provide. For instance, layers that provide mechanical reinforcement can be synthesized with composites of fabrics made from PET or fluoro polymers or UHMWPE or other biocompatible polymers as a reinforcement and/or aliphatic TPUs. Aromatic or modified TPUs can be used as a polymeric matrix. Layers for endothelialization and tissue ingrowth can utilize PET. Layers for resisting tissue ingrowth and pannus formation can be composed of biocompatible materials, such as (for example) TPUs, siloxanes, acrylics, UHMWPEs, and Fluoropolymers.

To synthesize multilayered covers, any of the variety methodologies described herein can be utilized, including electrospinning, three-dimensional printing, spray coating, dip coating, and lamination. Furthermore, any combination of methodologies in any order can be utilized.

As a layer is synthesized onto a prosthetic frame, a textile, a substrate, or another layer, it can be compressed to yield a thin layer membrane.

Generally, the frame/textile/substrate and synthesized layers can be inserted into a compression molding apparatus such that the layer can compressed. In some instances, the layer is compressed into a thickness of approximately 20-100 μm.

Synthesized cover layers can be further modified on its surface. Modifications can provide particular functions and/or enhance certain functions. Some modifications can promote resistance to tissue ingrowth and endothelialization. In some instances, a synthesized cover layer is fluorinated (especially TPU) or coated with fluorinated substance to resist tissue ingrowth and promote endothelialization. Likewise, treating a synthesized cover layer with plasma or corona will improve resistance to tissue ingrowth and promote endothelialization by imparting desired functionality through chain scission or grafting. Plasma and corona treatments can also improve adhesion of one layer with another via lamination or other means. Other modifications can attract tissue ingrowth and promote healing, which can help maintain implant in place in the long term and mitigating risks of embolism. For instance, a synthesized cover layer can be coated with amino acids (e.g., lysine or ornithine) or with proteins (e.g., collagen, fibronectin, or chitosan) to improve tissue ingrowth, endothelialization, and healing. A synthesized cover layer can also be treated with growth factors (e.g., VEGF) to improve endothelialization but limiting pannus overgrowth.

The surface topography can also be modified to yield particular functions. For instance, texturing a surface can improve adherence of a synthesized cover layer to the prosthetic frame, to a textile, or to another cover layer. Furthermore, pattern features, can improve endothelialization. Pattern features can be generated by, for example, texturing, embossing, or micropatterning. On the other hand, smoothening a cover layer to yield a more even surface topography can improve resistance to tissue ingrowth. Pattern features can be generated by (for example) lithography, photolithography, shaving, cutting, cauterizing, lasering, and aerosol spraying. Methods for smoothening can be performed by (for example) lasering, aerosol spraying, coating, or plasma treatment.

### Electrospinning Techniques to Yield Particular Properties

It is desired to generate prosthetic covers having particular properties that enhance its function. These enhanced functions can be achieved by synthesizing the cover in a particular manner. Some desirable properties are covers having low profile, controlled porosity, controlled hydrophilicity/hydrophobicity, functional layering, high push forces, superior edge quality, texturing, and biological responsiveness (e.g., interaction with the host). Electrospinning is an additive process that can be controlled to yield many particular properties that are beneficial for prosthetic covers.

A layer of electrospun fibers can be deposited on a prosthetic surface, a frame, a textile, a substrate, or another layer of electrospun fibers. If the electrospun fibers are deposited onto a textile or substrate, then the layer of electrospun fibers can be attached to a prosthetic surface or a frame with or without the textile/substrate. In some implementations, when fibers are spun onto a textile, the textile is also utilized as a cover layer on a prosthetic surface or frame and when fiber are spun onto a substrate, the layer of fibers are removed from the substrate when attached to the frame.

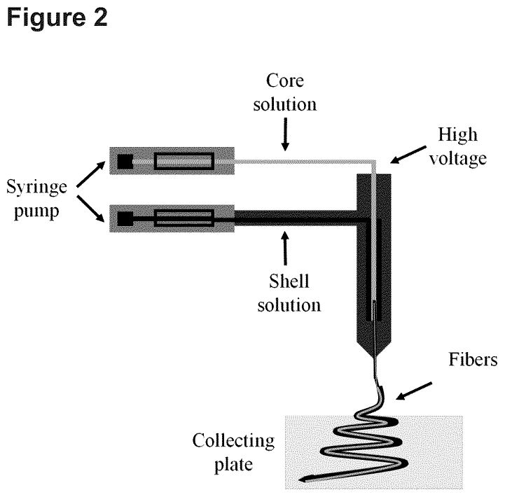

Fibers for electrospinning can comprise a core and a sheath surrounding the core. Typically, the inner core can provide mechanical properties (e.g., strength, push forces, and durability) while the outer sheath can provide various functionalities. A solution of inner core material can be charged with high voltage and then come into contact with the sheath solution to yield a mixed solution for depositing (FIG. 2). A positive or negative voltage can be utilized to generate negatively charged fibers or positively charged fibers, as is understood in the art. Upon depositing, charged fibers are formed and collected on a collecting material. A polarized material having an opposite charge of the fibers is utilized to attract the electrospun fibers. A collecting material (e.g., prosthetic surface, a frame, textile, or another layer of electrospun fibers) can be superimposed onto the polarized material, or alternatively the collecting material can be charged, for collecting the fibers. The collecting material can be a prosthetic surface, a frame, a textile, a substrate, or another layer of electrospun fibers.

A few polymers have been found to be useful in the synthesis of prosthetic covers. Generally, the polymer should be biocompatible and able to provide good electrospinning ability. In some implementations, the polymer utilized for the core is resolvable such that it breaks down over time after implementation. In some implementations, the material utilized for the core is nonresolvable, which can provide long term (or lifetime) integration after implantation. Resolvable polymers that can be utilized include (but are not limited to) silk fibroin, chitosan, polycaprolactone (PCL), poly(lactic-co-glycolic acid) (PGLA), poly (β-hydroxybutyrate-co-β-hydroxy valerate) (PHBV), and polyhydroxy butyrate (PHB). Nonresolvable polymers that can be utilized include (but are not limited to) polyolefins, polyethylene terephthalate (PET), thermoplastic polyurethane (TPU), expanded polytetrafluoroethylene (ePTFE), and polyvinylpyrrolidone (PVP). Polymers for use as the sheath can include (but are not limited to) a copolymer of polycarbonate TPU with siloxane, but depend on the functionality desired. Solvents that can be utilized include (but are not limited to) tetrahydrofuran (THF), dimethylacetamide (DMAc), hexafluoroisopropanol (HFIP), acetone, ethyl acetate, acetic acid, and trifluoroacetic acid.

Various polymers can be utilized based on their properties. For instance, PET is a well-known polymer with excellent biocompatibility. PCL is a biodegradable aliphatic polyester having a stiffness comparable to the native valve leaflet and thus useful in vascular prosthetics (e.g., replacement heart valves). Silk fibroin has very high biocompatibility and can resorb within the body over time. SF also has high permeability, especially for oxygen, water, and drug medications and has effective resistance against enzymatic degradation, rendering it a useful polymer in many situations.

In one example, a core polymer in solution can be negatively charged with a positive high voltage. The core polymer can be combined with a sheath polymer in solution that are dispelled out of a nozzle yield a jet of solution that converts into a fiber. The collecting material can be a prosthetic surface or frame, which can be mounted on a mandrel (or collecting drum) or on a flat surface having positively charged material (e.g., Aluminum). The positively charged material attracts the negatively charged polymer, which deposits on the prosthetic surface or frame.

The core polymer and sheath polymer solution can be released in a controlled manner (e.g., via a syringe and needle). The distance between from where the polymer solution is released and the collecting material, solvent concentration, polymer concentration, and the speed of the mandrel (when a mandrel is utilized) can be manipulated to control strand thickness and/or porosity. The longer the distance between the release of the polymer and the collecting material will result in the fibers being stretched into a smaller diameter. The distance between the release of the polymer can be up to about 200 cm.

Increasing the applied voltage will also resulted in stretching the polymer solution consistent with the charge repulsion in the jet, which will result in fibers having a smaller diameter. Care must be taken to not exceed a critical voltage, as this will result in beading (or droplet formation) of the polymer instead of fiber formation. The critical voltage is dependent on the polymer solution and thus can be optimized based on the parameters of the solution. Further, an increase in the flow rate increases the electric current and decreases the surface charge density, allowing the merging of electrospun fibers during their flight toward the collecting material. For fiber formation, the applied voltage can be between about 5 kV to 50 kV, dependent on the polymer.

The polymer solution should maintain an appropriate level of viscosity. When viscosity is too low, the applied voltage and surface tension cause the polymer fibers to break into fragments prior to reaching the collecting material. The fragments can cause the formation of bead droplets or beaded fibers.

Increasing polymer concentration increases viscosity and improves chain entanglement to form bead-less straight fibers. However, if the viscosity is too high, the flow of the polymer solution out of the syringe and needle will be hindered. Accordingly, the polymer concentration is typically between 5% and 25% w/v, but can vary dependent on the polymer.

A polymer solution will need a requisite amount of conductivity to have enough charge to form a Taylor cone. Conductivity can also affect fiber diameter. In some instances, a polymer solution will include KH₂PO₄, NaH₂PO₄, and/or NaCl at about 1% w/v to provide conductivity. In some instances, trialkylammonium chlorides can be added to polymer solution to decrease fiber diameter.

Humidity and temperature can also affect fiber formation. Humidity can alter the diameter of electrospun fibers, but is dependent on the polymer in solution. For instance, higher humidity will increase the diameter of cellulose acetate fibers but decrease the diameter of PVP fibers. Increases in temperature can cause solvent evaporation but the viscosity of the polymer solution also decreases. Thus, temperature should be controlled to keep viscosity and polymer concentration stable.

Porosity of a cover layer can be controlled by the thickness of the fibers and the amount of fiber deposited. For instance, higher porosity can be achieved depositing smaller diameter fibers and/or depositing less fibers such that they remain sparse and less overlapped.

Various treatments can improve the strength of electrospun fibers, each of which can be utilized in synthesis of one or more cover layers. For instance, plasma treatment activates free radicals on the substrate which then crosslinks and forms bonds with depositing layer or amongst chain of depositing nanofibers layers to increase the strength. The substrate can be a prosthetic frame, a textile, or another layer of deposited electrospun fibers. A plasma treatment on a cover layer can also texturize fiber surfaces, yield the fiber more hydrophobic, increase strength by crosslinking. Thus, plasma treatments can improve crosslinking of electrospun fibers within an electrospun layer, improve the bonding between electrospun fiber layers, improve the bonding between an electrospun layer and textile, and improve the bonding between an electrospun layer and prosthetic frame, each of which can result in an improved cover strength.

The hydrophobicity and hydrophilicity of a layer of electrospun fibers can be altered by plasma treatment. For instance, to increase hydrophobicity, CF₄ (1-2% in He) plasma can introduce and deposit fluorine groups on the fiber surface. Alternatively, to increase hydrophilicity, O₂ (1-2% in He) plasma can introduce and deposit oxygen free radicals that can attach to the polymeric fiber as alcohols, carbonyls, and carboxylic acids, yielding highly polar groups on the surface.

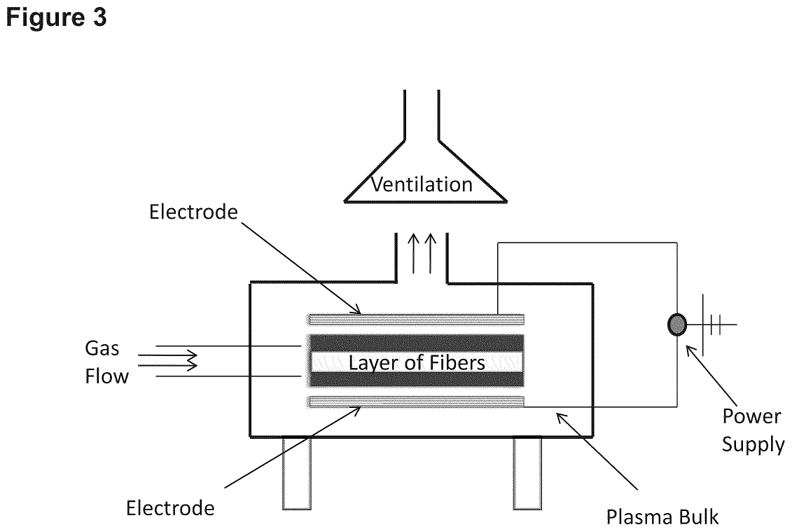

Provided in FIG. 3 is an example of a machine for performing plasma on a layer of electrospun fibers utilizing an atmospheric pressure plasma system. A layer of electrospun fibers is positioned between two electrodes within a chamber. Gas is provided to flow to and within the chamber, where a power supply is utilized to generate plasma bulk. The generated plasma treats the electrospun fibers.

Further, in some instances, a heat treatment and/or a repeated freeze-and-thaw treatment can increase the strength of a cover layer synthesized by electrospun fibers.

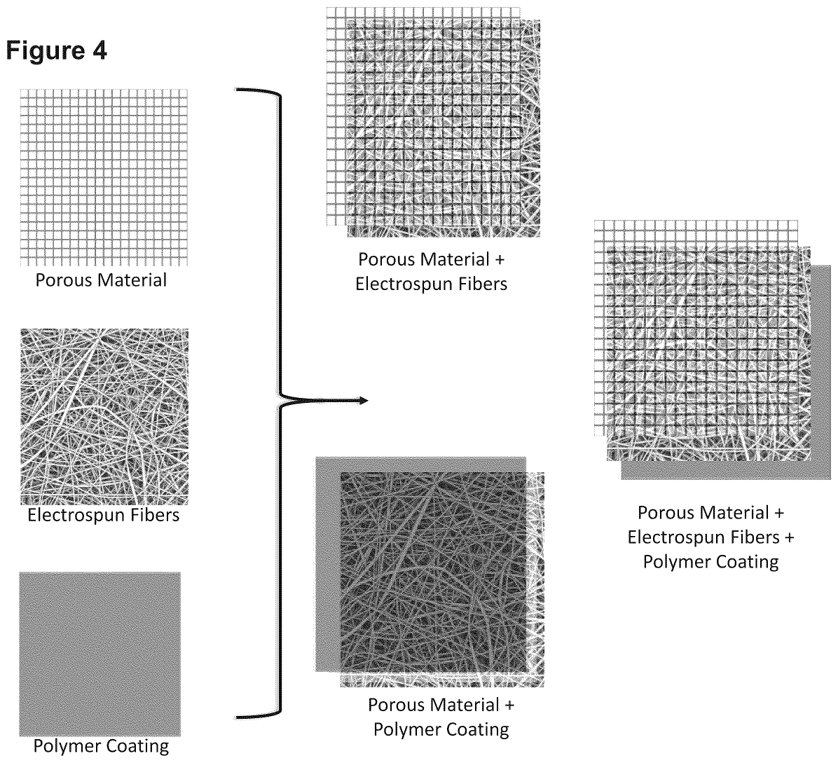

An electrospun fiber layer can be combined with one or more other coatings and/or textile layers, which can enhance some functionalities. For instance, a gauze-like or other porous textile can be attached to an electrospun fiber layer, which could enhance the mechanical properties of the fibers and also provide exposure of functional layers of the fibers via the porosity. In some instances, polymeric materials can be coated onto an electrospun fiber layer which can seal pores of the fiber layer and further reduce the ability of tissue ingrowth. Examples polymeric materials for coating include (but are not limited to) polyolefins, polyethylene terephthalate (PET), thermoplastic polyurethane (TPU), expanded polytetrafluoroethylene (ePTFE), siloxanes, and acrylics. In some instances, polymers are combined and/or blended. For example, a copolymer of polycarbonate TPU with siloxane or a blend of silica particles with TPU can be utilized as materials for coating. And in some instances, a porous material, such as a porous textile (e.g., the low-profile textiles described herein) can be attached to one side of an electrospun fiber layer and substantially impermeable and/or nonporous coat can be attached to the other side (FIG. 4).

A layer of electrospun fibers can be deposited as a cover on one or more surfaces of a prosthetic (e.g., an outer cover and an inner cover on a tubular prosthetic) a frame, a textile, or a substrate. To generate a cover, a prosthetic can be placed on a collecting material to attract the electrospun fibers. In some instances, a tubular prosthetic frame and collecting material are placed on a mandrel such that the electrospun fibers can deposit onto the prosthetic as the mandrel rotates, resulting in an outer cover attached to the prosthetic. To generate an inner cover on a tubular prosthetic, electrospun fibers can be deposited within the inner wall of the tubular prosthetic with a collecting material surrounding the outer wall of the prosthetic such that it attracts the electrospun fibers to the inner wall of the prosthetic. To deposit upon a textile, the textile can be placed on a collecting material to attract the electrospun fibers. In some instances, the textile and collecting material are placed on a mandrel such that the electrospun fibers can deposit onto the prosthetic as the mandrel rotates. After a layer of electrospun fibers is deposited, the textile can then be attached to a prosthetic by any means. When the electrospun fibers are deposited on a substrate, the layer of fibers can be removed from the substrate and utilized as needed. For instance, it can be combined with other layers and/or attached to a prosthetic.

In some instances, two or more covers can be deposited simultaneously on a single collecting material, which can be provided with the prosthetic on a flat surface or mandrel. For example, when creating a cover for a tubular prosthetic, two or more layers can be deposited simultaneously on a single mandrel to yield a single deposit of electrospun fibers. To do so, the prosthetic can be loaded onto mandrel or flat surface with the collecting material. The electrospun fibers can be deposited onto the outer wall of the prosthetic to form an external cover and also onto the mandrel adjacent to the frame to form an inner cover. The fibers can be deposited as a single deposit, with one portion of the cover on the frame and one portion of the off the frame and on the mandrel. When the frame and fibers are removed off the mandrel, the portion of fibers deposited on the mandrel can then be folded across an edge of the tubular frame and along the inner wall of the tubular frame to form an inner cover. This technique can be utilized to create one or more covers on prosthetic surfaces, a frame, or a textile in which a at least a portion of the cover can be synthesized by depositing electrospun fibers directly upon and adjacent to a surface, frame or textile and then folding the adjacently deposited electrospun fibers over an edge onto a second surface. Further, the electrospun fibers can be deposited such that the directly deposited electrospun have different properties than the adjacently deposited electrospun fibers. For example, in some instances, directly deposited cover is synthesized to have greater porosity than the adjacently deposited cover, or vice versa. And in some instances, the directly deposited cover is synthesized to have a greater profile than the adjacently deposited cover, or vice versa. In some instances, the electrospun fibers are deposited such that a directly deposited cover is configured to be in contact with tissue and the adjacently deposited cover is configured to be facing a vascular lumen, or vice versa.

As an example of generating a cover with multiple properties, a tubular valvular prosthetic having an expandable frame is considered. The tubular valvular prosthetic can be loaded onto a mandrel and a single deposit of electrospun fibers can deposited on a portion of the outer wall of the expandable frame to form an external cover and can further be deposited on collecting material adjacent to the frame. The expandable frame and deposited cover can be removed from the mandrel and the cover can be folded across the inflow edge, the outflow edge, or both the inflow and the outflow edges of the expandable frame. The folded over portion of the cover can span and be attached to at least a portion of the inner wall of the expandable frame to form the inner cover. In some instances, the properties of the portion of the cover deposited to form the external cover is synthesized to have different properties than the portion deposited to form the inner cover. For example, in some instances, the external cover is synthesized to have greater porosity than the inner cover. And in some instances, the external cover is synthesized to have a greater profile than the inner cover. Properties of valvular covers are described in greater detail below.

Electrospun fibers can also be utilized to generate leaflets for use in a replacement heart valve. To generate leaflets, a heart valve leaflet shaped mandrel can be used as collector and electrospun fibers can be deposited thereupon. Alternatively, a sheet of electrospun fiber can be deposited on a substrate and leaflets can be laser cut from the sheet of fibers.

### Fabricating Textiles with Low Profile

In addition to additive synthesis methods to generate covers with particular properties, textiles can be made into prosthetic covers to have advanced properties as well. Enhanced properties can yield various benefits for a cover. For instance, it is beneficial to produce a cover having a low profile and thus increase the ability to traverse through the body and reduce discomfort in minimally invasive procedures. To produce a low-profile cover, a textile can be fabricated with a low density of yarns, which can be further flattened. The textile can also be further textured to yield a smooth or rough surface to yield desired properties. The edges of a textile can be cut in a manner to yield high quality edges (e.g., to prevent pilling, tangling or unraveling).

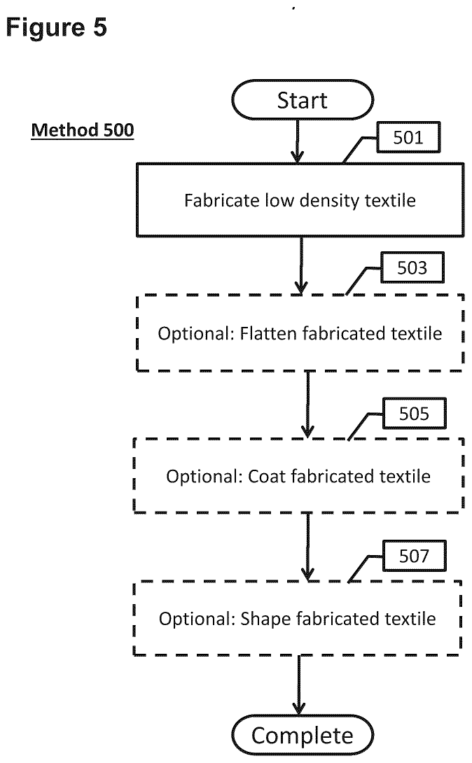

Provided in FIG. 5 is an example of a method to generate a textile with a shallow profile, which can be used as a cover for a prosthetic. Method 500 can begin by fabricating (501) a textile.

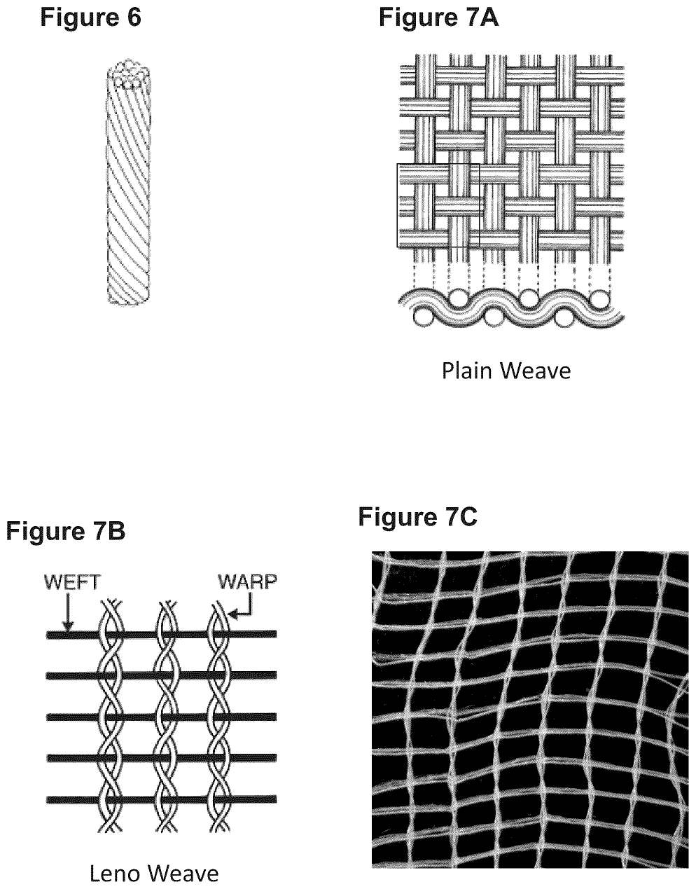

The multifilament yarn can consist of any whole number of yarn filaments from 2 filaments to 160 filaments that are twisted together (FIG. 6). In various implementations, the yarn consists of 5 filaments, the yarn consists of 6 filaments, the yarn consists of 7 filaments, the yarn consists of 8 filaments, the yarn consists of 9 filaments, the yarn consists of 10 filaments, the yarn consists of or about 12 filaments, the yarn consists of or about 15 filaments, the yarn consists of or about 18 filaments, the yarn consists of or about 20 filaments, the yarn consists of or about 22 filaments, the yarn consists of or about 25 filaments, the yarn consists of or about 30 filaments, the yarn consists of or about 35 filaments, the yarn consists of or about 40 filaments, the yarn consists of or about 45 filaments, the yarn consists of or about 50 filaments, the yarn consists of or about 60 filaments, the yarn consists of or about 70 filaments, the yarn consists of or about 80 filaments, the yarn consists of or about 90 filaments, the yarn consists of or about 100 filaments, the yarn consists of or about 110 filaments, the yarn consists of or about 120 filaments, the yarn consists of or about 130 filaments, the yarn consists of or about 140 filaments, the yarn consists of or about 150 filaments, or the yarn consists of or about 160 filaments. In regards to yarn filaments, “about” is meant to be ±5 filaments.

The multifilament yarn can be any appropriate size to yield a low-profile textile. Accordingly, in some instances, the yarn is between 5 and 40 denier. In various implementations, the yarn is or about 5 denier, the yarn is or about 10 denier, the yarn is or about 15 denier, the yarn is or about 20 denier, the yarn is or about 25 denier, the yarn is or about 30 denier, the yarn is or about 35 denier, or the yarn is or about 40 denier. In various implementations, the yarn is less than about 40 denier, the yarn is less than about 35 denier, the yarn is less than about 30 denier, the yarn is less than about 25 denier, the yarn is less than about 20 denier, the yarn is less than about 15 denier, or the yarn is less than about 10 denier. In regards to yarn denier, “about” is meant to be ±2.5 denier.

In some instances, the filament size can be between 0.20 denier and 3 denier. In various implementations, the filament is or about 0.20 denier, the filament is or about 0.25 denier, the filament is or about 0.5 denier, the filament is or about 0.75 denier, the filament is or about 1.0 denier, the filament is or about 1.25 denier, the filament is or about 1.5 denier, the filament is or about 1.75 denier, the filament is or about 2.0 denier, the filament is or about 2.25 denier, the filament is or about 2.5 denier, the filament is or about 2.75 denier, or the filament is or about 3.0 denier. In various implementations, the filament is less than about 3.0 denier, the filament is less than about 2.5 denier, the filament is less than about 2.0 denier, the filament is less than about 1.5 denier, the filament is less than about 1.0 denier, or the filament is less than about 0.5 denier. In regards to filament denier, “about” is meant to be ±0.125 denier.

The material of the filament can be a synthetic or naturally sourced filament. In some instances, the filament is composed of PET. In some instances, higher tenacity PET is desired, which can be defined being greater than 6 g/denier. Typical PET has a tenacity of about or less than 6 g/denier. In regards to PET tenacity, “about” is meant to be ±0.5 g/denier. Higher tenacity filaments allow for lower density of yarns per textile.

The twisting process of the filaments to yield a compact yarn should reduce the bulk of the filaments within it by keeping the bundle of filaments coherent. When a weaving process is used, warp yarn and/or weft yarn can be twisted. Generally, in some instances, the number of twists per inch should be between 3 turns per inch to 16 turns per inch. In various implementations, the yarn is about 3 turns per inch, about 4 turns per inch, about 5 turns per inch, about 6 turns per inch, about 7 turns per inch, about 8 turns per inch, about 9 turns per inch, about 10 turns per inch, about 11 turns per inch, about 12 turns per inch, about 13 turns per inch, about 14 turns per inch, about 15 turns per inch, or about 16 turns per inch. In regards to turns per inch, “about” is meant to be ±0.5 turns.

To fabricate the textile, a multifilament yarn can be woven, knitted, and/or braided. In some instances, the textile is woven cover for use on a prosthetic. While any orientation of the woven cover can be utilized, in some instances, the orientation is such that the multifilament yarns are positioned about 45-degrees (±15-degrees) relative to the longitudinal axis and/or latitudinal axis of the prosthetic. In one example of a valvular prosthetic, the woven cover is oriented such that that the multifilament yarns are positioned about 45-degrees (±15-degrees) relative to the valve axis (i.e., the intended direction of blood flow). This orientation can help in the crimping process of the prosthetic when used within a transcatheter system, decreasing the overall profile of the prosthetic during delivery.

In some instances, a woven textile has between about 150 to about 200 ends per inch (EPI) and/or between 150 to about 200 picks per inch (PPI). In various implementations, a woven textile is or about 150 EPI, a woven textile is or about 155 EPI, a woven textile is or about 160 EPI, a woven textile is or about 165 EPI, a woven textile is or about 170 EPI, a woven textile is or about 175 EPI, a woven textile is or about 180 EPI, a woven textile is or about 185 EPI, a woven textile is or about 190 EPI, a woven textile is or about 195 EPI, or a woven textile is or about 200 EPI. In various implementations, a woven textile is between about 145 EPI and about 165 EPI, a woven textile is between about 155 EPI and about 165 EPI, a woven textile is between about 165 EPI and about 185 EPI, a woven textile is between about 175 EPI and about 195 EPI, or a woven textile is between about 185 EPI and about 205 EPI. In various implementations, a woven textile is or about 150 PPI, a woven textile is or about 155 PPI, a woven textile is or about 160 PPI, a woven textile is or about 165 PPI, a woven textile is or about 170 PPI, a woven textile is or about 175 PPI, a woven textile is or about 180 PPI, a woven textile is or about 185 PPI, a woven textile is or about 190 PPI, a woven textile is or about 195 PPI, or a woven textile is or about 200 PPI. In various implementations, a woven textile is between about 145 PPI and about 165 PPI, a woven textile is between about 155 PPI and about 165 PPI, a woven textile is between about 165 PPI and about 185 PPI, a woven textile is between about 175 PPI and about 195 PPI, or a woven textile is between about 185 PPI and about 205 PPI. In regards to woven textile density, “about” is meant to be ±2.5 EPI or ±2.5 PPI.

In some instances, a woven textile has between about 100 to about 150 ends per inch (EPI) and/or between 100 to about 150 picks per inch (PPI), which can be woven with high tenacity yarn (e.g., greater than 6 g/denier). In various implementations, a woven textile is or about 100 EPI, a woven textile is or about 105 EPI, a woven textile is or about 110 EPI, a woven textile is or about 115 EPI, a woven textile is or about 120 EPI, a woven textile is or about 125 EPI, a woven textile is or about 130 EPI, a woven textile is about 135 EPI, a woven textile is about 140 EPI, a woven textile is or about 145 EPI, a woven textile is or about 150 EPI. In various implementations, a woven textile is between about 95 EPI and about 115 EPI, a woven textile is between about 105 EPI and about 125 EPI, a woven textile is between about 115 EPI and about 135 EPI, a woven textile is between about 125 EPI and about 145 EPI, or a woven textile is between about 135 EPI and about 155 EPI. In various implementations, a woven textile is or about 100 PPI, a woven textile is or about 105 PPI, a woven textile is or about 110 PPI, a woven textile is or about 115 PPI, a woven textile is or about 120 PPI, a woven textile is or about 125 PPI, a woven textile is or about 130 PPI, a woven textile is or about 135 PPI, a woven textile is or about 140 PPI, a woven textile is or about 145 PPI, a woven textile is or about 150 PPI. In various implementations, a woven textile is between about 95 PPI and about 115 PPI, a woven textile is between about 105 PPI and about 125 PPI, a woven textile is between about 115 PPI and about 135 PPI, a woven textile is between about 125 PPI and about 145 PPI, or a woven textile is between about 135 EPI and about 155 EPI. In regards to woven textile density, “about” is meant to be ±2.5 EPI or ±2.5 PPI.

In some instances, a woven textile has between about 60 to about 100 ends per inch (EPI) and/or between 60 to about 100 picks per inch (PPI), which can be woven with high tenacity yarn (e.g., greater than 6 g/denier). In various implementations, a woven textile is or about 60 EPI, a woven textile is or about 65 EPI, a woven textile is or about 70 EPI, a woven textile is or about 75 EPI, a woven textile is or about 80 EPI, a woven textile is or about 85 EPI, a woven textile is or about 90 EPI, a woven textile is or about 95 EPI, or a woven textile is or about 100 EPI. In various implementations, a woven textile is between about 55 EPI and about 75 EPI, a woven textile is between about 65 EPI and about 85 EPI, a woven textile is between about 75 EPI and about 95 EPI, or a woven textile is between about 85 EPI and about 105 EPI. In various implementations, a woven textile is or about 60 PPI, a woven textile is or about 65 PPI, a woven textile is or about 70 PPI, a woven textile is or about 75 PPI, a woven textile is or about 80 PPI, a woven textile is or about 85 PPI, a woven textile is or about 90 PPI, a woven textile is or about 95 PPI, or a woven textile is or about 100 PPI. In various implementations, a woven textile is between about 55 PPI and about 75 PPI, a woven textile is between about 65 PPI and about 85 PPI, a woven textile is between about 75 PPI and about 95 PPI, or a woven textile is between about 85 PPI and about 105 PPI. In regards to woven textile density, “about” is meant to be ±2.5 EPI or ±2.5 PPI.

In some instances, a woven textile has between about 30 to about 60 ends per inch (EPI) and/or between 30 to about 60 picks per inch (PPI), which can be woven with high tenacity yarn (e.g., greater than 6 g/denier). In various implementations, a woven textile is or about 30 EPI, a woven textile is or about 35 EPI, a woven textile is or about 40 EPI, a woven textile is or about 45 EPI, woven textile is or about 50 EPI, a woven textile is or about 55 EPI, or a woven textile is or about 60 EPI. In various implementations, a woven textile is between about 25 EPI and about 45 EPI, a woven textile is between about 35 EPI and about 55 EPI, or a woven textile is between about 45 EPI and about 65 EPI. In various implementations, a woven textile is or about 30 PPI, a woven textile is or about 35 PPI, a woven textile is or about 40 PPI, a woven textile is or about 45 PPI, a woven textile is or about 50 PPI, a woven textile is or about 55 PPI, or a woven textile is or about 60 PPI. In various implementations, a woven textile is between about 25 PPI and about 45 PPI, a woven textile is between about 35 PPI and about 55 PPI, or a woven textile is between about 45 PPI and about 65 PPI. In regards to woven textile density, “about” is meant to be ±2.5 EPI or ±2.5 PPI.

To fabricate woven textile, the weaving machine or loom can help ensure the yarn is kept separate during the weaving process by choosing a machine or loom with an appropriate number of reed dents per inch. The desired EPI density of the fabricated textile should be equal to the reeds per dent of the loom such that only a single warp is within each dent. For example, to generate a woven textile having 40 EPI, the machine or loom should have 40 reed dents per inch with a single warp within each dent; or to generate a woven textile having 120 EPI, the machine or loom should have 120 reed dents per inch, with a single warp within each dent. Having a single warp per dent ensures an even spacing between the ends and picks while keeping yarn to yarn friction to a minimum. It should be understood that other methods besides the example described can be utilized to achieve the appropriate density desired.

A woven textile is to have adequate suture retention strength. In some implementations, a textile has a suture retention strength that is between about 5 to about 25 N. In various embodiments, the suture retention strength of a textile is about 5 N, the suture retention strength of a textile is about 10 N, the suture retention strength of a textile is about 15 N, the suture retention strength of a textile is about 20 N, or the suture retention strength of a textile is about 25 N. Further, to ensure maximum interlacement, the woven textile can be a plain weave (FIG. 7A) and/or a leno weave (FIG. 7B). In some implementations, a leno weave is used to prevent yarns from shifting in the woven textile, which may help retain the integrity of the textile. Provided in FIG. 7C is an example of leno-woven fabric.

In some instances, a multifilament yarn can be weft knitted or warp knitted to fabricate a textile. A weft knitted or warp knitted textile would provide stretchability in radial direction, which can be beneficial for compliance while a prosthetic (e.g., replacement valve) undergoes crimping and/or expansion. Stretchability can help prevent constructional damage to the prosthetic cover during the crimping process for inserting within a transcatheter and during the expansion process when implanted. This stretchability can range from 30 to 100%, or can be measured as an expansion ratio. In some implementations, the expansion ratio is between about 1.0:1.0 to about 1.0:1.7. In various embodiments, the expansion ratio is about 1.0:1.0, the expansion ratio is about 1.0:1.1, the expansion ratio is about 1.0:1.2, the expansion ratio is about 1.0:1.3, the expansion ratio is about 1.0:1.4, the expansion ratio is about 1.0:1.5, the expansion ratio is about 1.0:1.6, or the expansion ratio is about 1.0:1.7. In regards to expansion ratio, “about” is meant to be ±0.05 (e.g., about 1.0:1.1 is equivalent to 1.0:1.05 to 1.0:1.15).

A weft knitted or warp knitted textile can be knitted in a tubular shape, which can be utilized as an inner cover, an outer cover, or both an inner cover and an outer cover on a tubular prosthetic. The diameter of a tubular weft knitted or warp knitted textile will be dependent on the diameter of the tubular prosthetic and the stretchability of the textile. In one example, a tubular knitted textile is for a valvular prosthetic, and the diameter of the tubular knitted textile is between 28 mm and 32 mm for a 30 mm prosthetic. An outer cover can have a larger diameter than an inner cover, or the same diameter, as may be dependent on stretchability. In some implementations, a tubular knitted textile is knitted to comprise closed ends at each end of the tubular shape. Closed ends can have the benefit of preventing laddering.

In instances that the textile is weft knitted or warp knitted, the density can range from about 10 to 50 courses per inch (CPI) and from about 20 to 60 wales per inch (WPI). In various implementations, the density of a knitted textile is or about 10 CPI, the density of a knitted textile is or about 15 CPI, the density of a knitted textile is or about 20 CPI, the density of a knitted textile is or about 25 CPI, the density of a knitted textile is or about 30 CPI, the density of a knitted textile is or about 35 CPI, the density of a knitted textile is or about 40 CPI, the density of a knitted textile is or about 45 CPI, or the density of a knitted textile is about 50 CPI. In various implementations, a knitted textile is between about 5 CPI and about 25 CPI, a knitted textile is between about 15 CPI and about 35 CPI, a knitted textile is between about 25 CPI and about 45 CPI, or a knitted textile is between about 35 CPI and about 55 CPI. In various implementations, the density of a knitted textile is or about 20 WPI, the density of a knitted textile is or about 25 WPI, the density of a knitted textile is or about 30 WPI, the density of a knitted textile is or about 35 WPI, the density of a knitted textile is or about 40 WPI, the density of a knitted textile is or about 45 WPI, the density of a knitted textile is or about 50 WPI, the density of a knitted textile is or about 55 WPI, or the density of a knitted textile is or about 60 WPI. In various implementations, a knitted textile is between about 15 WPI and about 35 WPI, a knitted textile is between about 25 CPI and about 45 EPI, a knitted textile is between about 35 WPI and about 55 WPI, or a knitted textile is between about 45 WPI and about 65 WPI. In regards to knitted textile density, “about” is meant to be ±2.5 CPI or ±2.5 WPI.

In some instances, a multifilament yarn can be braided to fabricate a textile. A braided textile could be provided as flat sheet or as a seamless tube, which would be beneficial for use with tubular shaped prosthetics. While any orientation of the braided cover can be utilized, in some instances, the orientation is such that the multifilament yarns are positioned about 45-degrees (±15-degrees), or about −45-degrees (±15-degrees), relative to the longitudinal axis and/or latitudinal axis of the prosthetic. In one example of a valvular prosthetic, the braided cover is oriented such that the multifilament yarns are positioned about 45-degrees (±15-degrees), or about −45-degrees (±15-degrees), relative to the valve axis (i.e., the intended direction of blood flow). This orientation can help ensure compliance of the textile during the crimping process for inserting a prosthetic with braided cover within a transcatheter and during the expansion process when it is implanted. In instances in which a textile is a braided sheet or a braided tube, the density of the textile can range between about 30 to 120 picks per inch (PPI). In various implementations, the density of a braided textile is or about 30 PPI, the density of a braided textile is or about 40 PPI, the density of a braided textile is or about 50 PPI, the density of a braided textile is or about 60 PPI, the density of a braided textile is or about 70 PPI, the density of a braided textile is or about 80 PPI, the density of a braided textile is or about 90 PPI, the density of a braided textile is or about 100 PPI, the density of a braided textile is or about 110 PPI, or the density of a braided textile is or about 120 PPI. In various implementations, a braided textile is between about 25 PPI and about 45 PPI, a braided textile is between about 35 PPI and about 55 PPI, a braided textile is between about 45 PPI and about 65 PPI, a braided textile is between about 55 PPI and about 75 PPI, a braided textile is between about 65 PPI and about 85 PPI, a braided textile is between about 75 PPI and about 95 PPI, a braided textile is between about 85 PPI and about 105 PPI, a braided textile is between about 95 PPI and about 115 PPI, or a braided textile is between about 105 PPI and about 125 PPI. In regards to braided textile density, “about” is meant to be ±5 PPI. In instances in which a seamless tubular braided textile is to be utilized with as a cover for valvular prosthetic, the diameter can be adjusted based on the valve size. In one example, a tubular braided textile is for a valvular prosthetic, and the diameter of the tubular braided textile is between 28 mm and 32 mm for a 30 mm prosthetic. An outer cover can have a larger diameter than an inner cover, or the same diameter, as may be dependent on stretchability.

A textile should be fabricated to yield a low profile, having a thickness between 20 μm and 100 μm, which can result in prosthetics having covers with low profiles. To yield a textile with a low profile but maintain strength and durability, the fabrication process should balance yarn denier, yarn density, and yarn tenacity. For woven or braided textiles, it has been found that higher tenacity yarn with denier between 10 and 25, having between 15 and 25 filaments, with about 30 to 100 EPI and PPI yielded a textile between 30 and 50 microns with requisite properties to be utilized within a cover for a prosthetic. Likewise, a tubular knit with higher tenacity yarn with denier between 5 and 15, having between 15 and 25 filaments, with about 25 to 50 WPI and CPI yielded a textile between 50 and 80 microns with requisite properties to be utilized within a cover for a prosthetic. The textiles could further be heat stabilized, condensed and/or flattened, each of which further reduce textile profile.

After fabrication, the textile can be further processed. In some implementations, the textile is scoured or otherwise cleaned. In some implementations, the textile is stabilized, which can be performed by a heat treatment.

Returning back to FIG. 5 Method 500 optionally further flattens (503) the textile. Flattening can provide various benefits, including (but not limited to) reducing the profile of the textile and smoothening the textile. A flattening process can reduce a profile of a fabricated textile 5% to 80%. When the textile has a density of yarns in the rages described herein, the profile can be substantially reduced. Any flattening process that provides pressure (and optionally heat) can be utilized, such as (for example) condensing, continuous roll calendaring, flat heat press, or any combination of flattening processes.

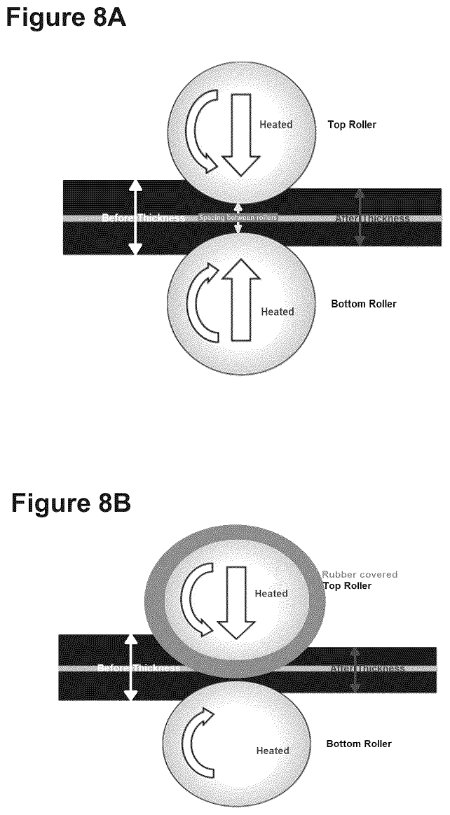

FIGS. 8A and 8B provide each provide an example of continuous roll calendaring. As shown in the figures, two stainless rollers, which can be covered with semi-pliable material such as rubber (FIG. 8B). The textile is feed into and between the two rollers. Because the spacing between the two rollers is less than the starting profile of the textile, the profile is reduced as it passes through the rollers. The rollers can be heated, which can assist in the flattening process.

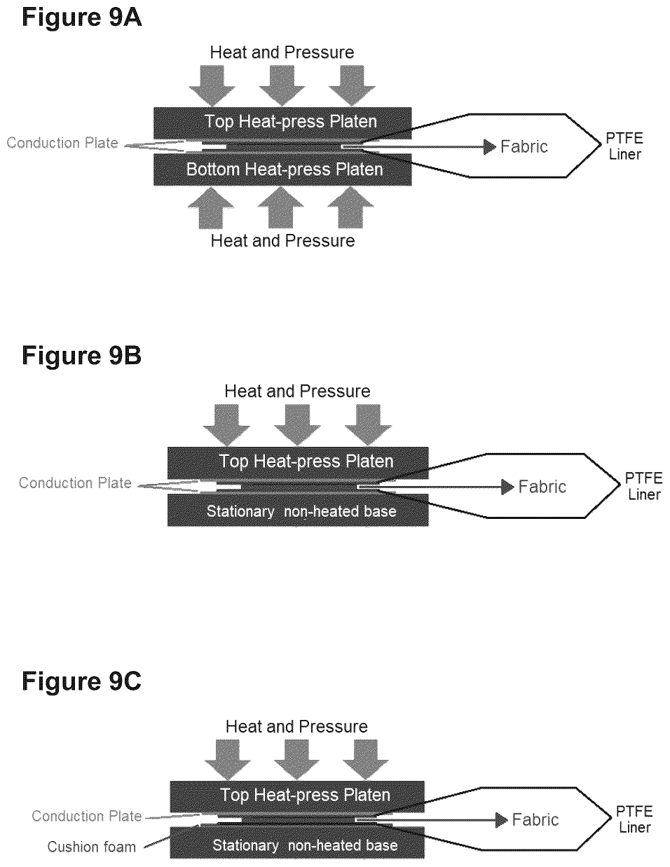

FIGS. 9A, 9B, and 9C each provide an example of flat heat press. As shown in the figures, a top heated platen used to apply heat and pressure by pressing down on a textile that is in between the top heated platen and base.

The base can be also be a heated platen (FIG. 9A) or a stationary non-heated base (FIGS. 9B and 9C). A stationary non-heated base can also be lined with a pliable substance such as (for example) cushion foam (FIG. 9C). By adding heat and pressure via the one or two platens, the textile is flattened out.

Method 500 can also optionally coat (503) the textile. Coating a solid layer can transform the textile into a substantially impermeable and/or nonporous textile. Any coating technique can be utilized, such as (for example) spray coating, dip coating, and lamination. In some implementations, ultrasonic coating is utilized.

Materials that can be utilized for coating the textile include (but are not limited to) polyolefins, polyolefins, polypropylene (PP), polyethylene terephthalate (PET), thermoplastic polyurethane (TPU), expanded polytetrafluoroethylene (ePTFE), siloxanes, and acrylics. In some instances, polymers are combined and/or blended. For instance, TPU and various siloxanes can be blended or various copolymers can be used. For example, a copolymer of polycarbonate TPU with siloxane, a blend of silica particles with TPU or fluorinated TPU and PET can be utilized as materials for coating. TPUs can also be fluorinated through coatings or with additives.

Method 500 can also optionally shape (505) the textile. Shaping can be performed on coated or uncoated textiles. To shape the textile, the textile can be cut into an appropriate size shape, which can be performed via any appropriate cutting technique such as (for example) die cutting or laser cutting, or any combination of cutting techniques. Further, the textile can be shaped by folding, bending, rolling, or otherwise converting the flat textile into non-flat shape. In some instances, the textile is shaped into a tube, which is useful for use as a cover for a prosthetic (e.g., valvular prosthetic). The shaped textile can be stitched or otherwise sealed on the edges, which can help the textile remain shaped. In some implementations, ultrasonic spraying is utilized to seal the edges. In some implementations, the material that is used to seal the edges of the shaped textile is the same as the material for coating the textile.

While specific examples of a method to fabricate a textile with a low profile is described above, one of ordinary skill in the art can appreciate that various steps of the method can be performed in different orders and that certain steps may be optional according to various implementations. As such, it should be clear that the various steps of the method could be used as appropriate to the requirements of specific applications. Furthermore, any of a variety of methods to fabricate a textile with a low profile appropriate to the requirements of a given application can be utilized in various implementations.

### Covers for Valvular Devices

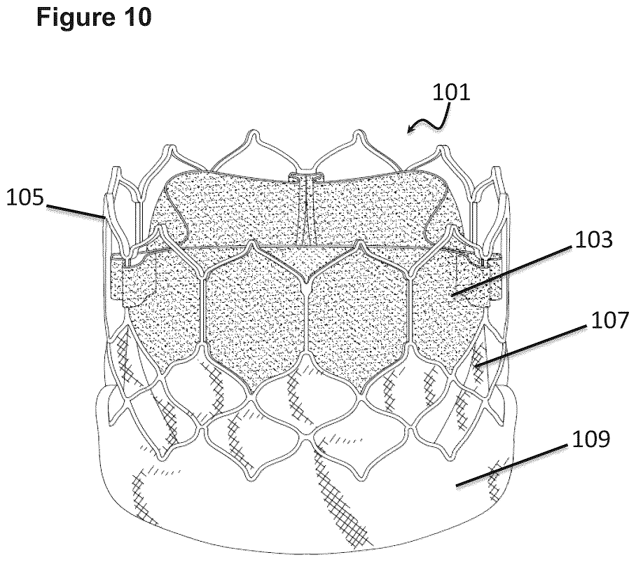

Synthesized covers can be utilized in conjunction with a valvular prosthetic. Provided in FIG. 10 is an example of a valvular prosthetic 101 for replacing a native heart valve. The valvular prosthetic 101 can be delivered via a transcatheter. The prosthetic can be crimped into a confined profile such that it can fit within a transcatheter delivery system. Generally, valvular prosthetic 101 comprises a set of leaflets 103 to perform the valve function, a frame 105 for supporting the leaflets, and an inner cover 107 to ensure proper blood flow through the valve. The valvular prosthetic can further comprise an outer skirt 109 to mitigate perivalvular leakage and/or protect the frame from the surrounding tissue or protect the surrounding tissue from the frame. The valvular prosthetic can further comprise an anchor or other means for securing the valve at the site of implantation.

Inner cover 107 and/or outer skirt 109 can be synthesized by the processes as described herein. Accordingly, inner cover 107 and/or or outer skirt 109 can be synthesized by electrospinning, three-dimensional printing, spray coating, dip coating, lamination, weaving, knitting, braiding, and any combination thereof. In some instances, inner cover 107 and/or outer skirt 109 can be directly applied to frame 105 without sutures or an adhesive. And in some instances, inner cover 107 and/or outer skirt 109 can be synthesized on a temporary substrate then processed and attached to frame 105 with suture or an adhesive. It should be understood that various instances can include a synthesized inner cover 107 and lack outer skirt 109 or alternatively can include a synthesized outer skirt 109 and lack inner cover 107. In some instances, inner cover 107 covers the entire luminal face of frame 105. In some instances, luminal cover 107 covers a portion but not the entire internal face of tubular frame 105. In some instances, outer skirt 109 covers the entire outer face of frame 105. In some instances, outer skirt 109 covers a portion but not the entire outer face of frame 105.

Various materials can be utilized to synthesize inner cover 107 and/or or outer skirt 109. In some instances, inner cover 107 and/or or outer skirt 109 comprise one or more of the following materials: silk fibroin, chitosan, polycaprolactone (PCL), poly(lactic-co-glycolic acid) (PGLA), poly (β-hydroxybutyrate-co-β-hydroxy valerate) (PHBV), polyhydroxy butyrate (PHB), polyolefins, polyethylene terephthalate (PET), thermoplastic polyurethane (TPU), expanded polytetrafluoroethylene (ePTFE), polyvinylpyrrolidone (PVP)and siloxanes. In some instances, inner cover 107 and/or or outer skirt 109 comprise a blend of polymers, such as (for example) TPU blended with siloxane.

Internal cover 107 and/or or outer skirt 109 can be a textile. In some instances, the density of inner cover 107 and/or or outer skirt 109 is woven textile between 40-200 EPI and 40-200 PPI. In some instances, inner cover 107 and/or or outer skirt 109 has a low profile. In some instances, inner cover 107 and/or or outer skirt 109 has a thickness of about 10-100 μm. In various instances, inner cover 107 and/or or outer skirt 109 has a thickness less than 100 μm, inner cover 107 and/or or outer skirt 109 has a thickness less than 80 μm, inner cover 107 and/or or outer skirt 109 has a thickness less than 60 μm, or inner cover 107 and/or or outer skirt 109 has a thickness less than 40 μm, or inner cover 107 and/or or outer skirt 109 has a thickness less than 20 μm. In some instances, the textile is fabricated in accordance with Method 500 as shown in FIG. 5. In some instances, the textile is fabricated in accordance with any of the methods described within U.S. Pat. Pub. No. 2019/0351099, the disclosure of which is herein incorporated by reference in its entirety.

Inner cover 107 and/or or outer skirt 109 can comprise a low profile. In some instances, it may be beneficial to keep the profile of inner cover 107 and/or outer skirt 109 low or ultralow (e.g., less than 40 μm). Keeping the profile of inner cover 107 low can be beneficial when the prosthetic valve is to be delivered via transcatheter delivery system, such that the overall profile of the prosthesis to be delivered within the delivery system is reduced. Reduction of profile can be achieved by reducing the number of layers synthesized, reducing the profile of layer synthesized, reducing the profile of a textile fabricated, flattening (or calendaring) or a layer, or other any other means that results in a low profile.

In some instances, one of outer skirt 109 or inner cover 107 has a lesser profile than the other. In some instances, outer skirt 109 has a lesser profile than inner cover 107. In some instances, inner cover 107 has a lesser profile than outer skirt 109. By fabricating one of the outer skirt or the inner cover to have a lesser profile, an overall reduced profile of the prosthetic may be achieved, improving the crimped profile for transcatheter delivery. There may be benefits of having differing profiles for an inner cover and an outer skirt. For example, a thicker inner cover may be of benefit to yield a smoother surface, which can decrease tissue ingrowth or pannus formation on within a vascular lumen. And a thicker outer skirt may be of benefit to prevent perivalvular leakage around the prosthetic valve. Accordingly, in some instances, an inner cover 107 has a greater profile than outer skirt 109 and in some instances, outer skirt 109 has a greater profile than inner cover 107. In some instances, inner cover 107 comprises a textile have a greater profile than outer skirt 109. In some instances, outer skirt 109 comprises a textile have a greater profile than inner cover 107.

In some implementations, outer skirt 109 comprises a woven textile with about 30 PPI to about 60 PPI and about 30 EPI to about 60 EPI, and inner cover 107 comprises a woven textile with about 60 PPI to about 100 PPI and about 60 EPI to about 100 EPI. In some implementations, outer skirt 109 comprises a woven textile with about 40 PPI and about 40 EPI, and inner cover 107 comprises a woven textile with about 80 PPI and about 80 EPI. In some implementations, outer skirt 109 comprises a woven textile with about 60 PPI to about 100 PPI and about 60 EPI to about 100 EPI, and inner cover 107 comprises a woven textile with about 30 PPI to about 60 PPI and about 30 EPI to about 60 EPI. In some implementations, outer skirt 109 comprises a woven textile with about 80 PPI and about 80 EPI, and inner cover 107 comprises a woven textile with about 40 PPI and about 40 PPI.

In some implementations, outer skirt 109 and inner cover 107 each comprise a knitted textile of about 10 CPI to about 50 CPI and 20 WPI to about 60 WPI, and the outer skirt has a lesser profile than inner cover. In some implementations, outer skirt 109 and inner cover 107 each comprise a knitted textile of about 10 CPI to about 50 CPI and 20 WPI to about 60 WPI, and the inner cover has a lesser profile than outer skirt.

The one or more layers of inner cover 107 and/or outer skirt 109 can have particular functionality. For example, the layer most near to frame 105 of inner cover 107 and/or outer skirt 109 can have properties for adherence to the frame. The most external layer (or layers) of outer skirt 109 can have properties for interacting with the host biology. And one or more layers therebetween can have properties for mechanical support.

It is beneficial for inner cover 107 to be resistant to tissue ingrowth and endothelialization to prevent occlusion of blood flow. Inner cover 107 can thus have properties that resist tissue ingrowth and endothelialization. In some situations, the inner cover 107 comprises fluoropolymers or other polymers that resist tissue ingrowth. In some instances, inner cover 107 is fluorinated or treated with a fluorinated substance. In some instances, inner cover 107 is treated with plasma or corona. Furthermore, the topology of the inner cover 107 can be smoothened. Methods for smoothening can be performed by (for example) lasering, aerosol spraying, coating, or plasma treatment.

It can also be beneficial for a prosthetic valve to integrate within the l°Cal environment of the implantation site. To improve integration, outer skirt 109 can have properties that promote tissue ingrowth and endothelialization. In some instances, outer skirt 109 comprises a biocompatible polymer, such as (for example) TPUs, siloxanes, acrylics, UHMWPEs, or PET. In some instances, outer skirt 109 comprises a coating of endothelialization-inducing biologic such as (for example) amino acids (e.g., lysine or ornithine), saccharides (e.g., hyaluronic acid, fibronectin, chitosan), structural proteins (e.g., collagen, elastin), growth factors (e.g., VEGF), and combinations thereof.

To further improve tissue ingrowth and endothelialization, outer skirt 109 can include a one or more pattern features (e.g., surface that is modified and/or texturized, embossed, or micropatterned). Line-patterning may influence the regeneration of a healthy endothelial cell monolayer of human vascular vein endothelial cells and human cardiac microvascular endothelial cells. (See e.g., Ding Y, et al. “Directing vascular cell selectivity and hemocompatibility on patterned platforms featuring variable topographic geometry and size.” ACS Appl Mater Interfaces 2014;6:12062-12070; and Pacharra S, et al. “Surface patterning of a novel PEG-functionalized poly-L-lactide polymer to improve its biocompatibility: Applications to bioresorbable vascular stents.” J Biomed Mater Res B Appl Biomater. 2019;107(3): 624-634; the disclosures of which are hereby incorporated by reference in their entireties.) Outer skirt 109 can be modified with line patterning to improve biocompatibility. Pattern features can be generated by (for example) lithography, photolithography, shaving, cutting, cauterizing, lasering, and aerosol spraying.

The pattern features can be multi-dimensional structures and/or patterns including (but not limited to) two dimensional (2D), three dimensional (3D), 2D and 3D, that may aid in reendothelization. The pattern features can include various shapes including (but not limited to) groves, pillars, pores, ridges, waves, and/or any other pattern shown to have the desired biological effect including (but not limited to) reendothelization and healing. As can readily be appreciated, any of a variety of patterns can be utilized as appropriate to the requirements of specific applications. In some instances, outer skirt 109 can include pattern features with periodic and/or random structures including (but not limited to) grooves, dimples, fishbone structure and squares that can enhance cellular adhesion, migration and proliferation. In some instances, at least one dimension of a pattern and/or structure can range from micro-scale (from about 1 μm to about 999 μm) to nano-scale (from about 10 nm to about 999 nm). A pattern feature on outer skirt 109 may enable the cover to be anisotropic.

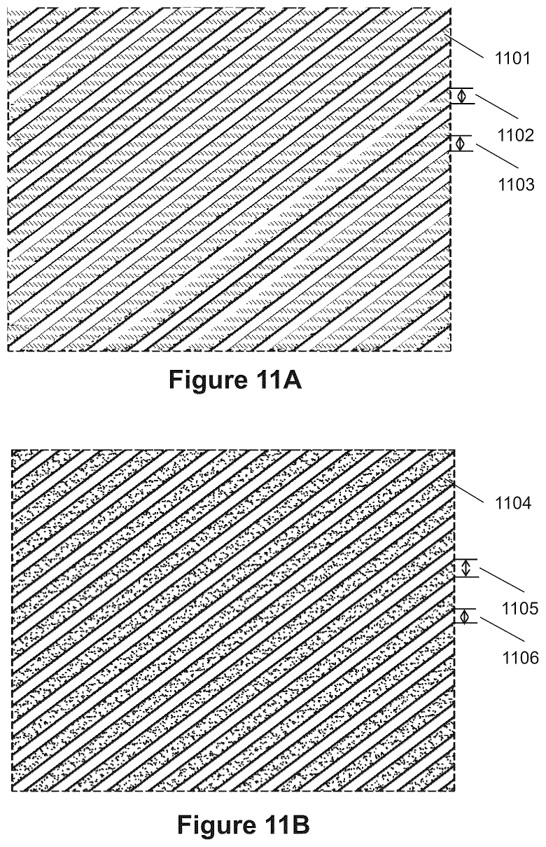

Pattern features can be micropatterns created on outer covers and skirts of prosthetic devices. Examples of micropattern features include parallel rows, such as illustrated in FIGS. 11A and 11B. Such rows can be created by ablation or subtraction of the material, e.g., troughs or trenches (e.g., FIG. 11A) or deposition or addition of material, creating ridges on the material (e.g., FIG. 11B). The dimensions between such rows can be of any suitable height (or depth), width, and spacing suitable for encouraging, enhancing, and/or promoting endothelization. In ablated examples, such as troughs 1101 illustrated in FIG. 11A, the depth ranges from about 1 μm to about 10 μm, while the width of the troughs 1102 from about 5 μm to about 20 μm is suitable for cellular implantation. The spacing between troughs 1103 can vary to allow cellular growth and/or allow for flexibility of the underlying material, and can be between about 5 μm and about 20 μm. Additionally, in deposited examples, such as ridges 1104 illustrated in FIG. 11B, the height of such ridges ranges from about 1 μm to about 10 μm, while the width of the ridges 1105 from about 1 μm to about 5 μm is suitable for cellular implantation. Spacing between ridges 1106 can vary to allow cellular growth and/or allow for flexibility of the underlying material, and can be between about 5 μm and about 20 μm. While FIGS. 11A and 11B illustrate linear rows, various implementations utilize zigzags, waves, sinusoids, and/or another other patterning of trenches and/or ridges on the material that promotes, encourages, or enhances reendothelization, and/or provides desired properties (e.g., rigidity, flexibility, etc.) to the underlying material.

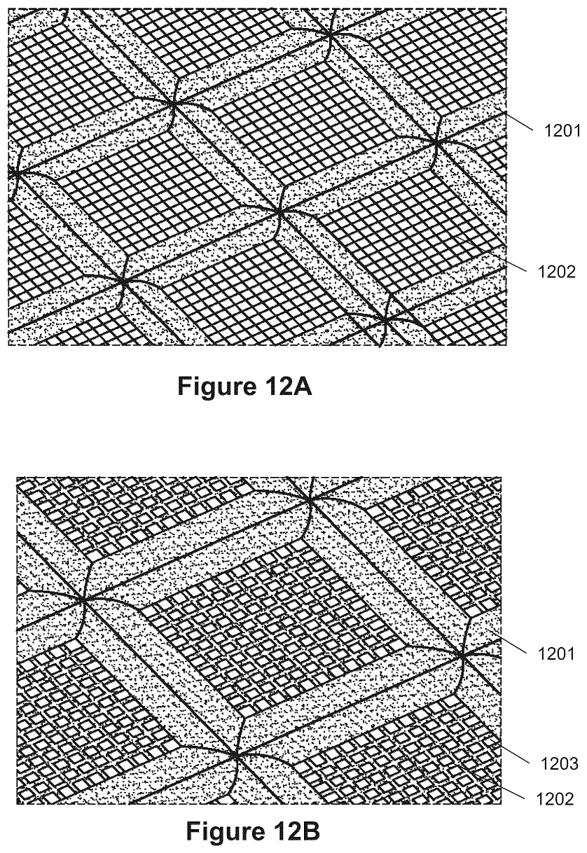

Pattern features can also comprise multidimensional and/or multilayered patterning. In some instances, a smaller pattern (e.g., nanopatterns) can be combined within a larger size pattern (e.g., micropattern). An example of such patterning is shown in FIGS. 12A and 12B. FIG. 12A illustrates a “chessboard” pattern, where a larger grid 1201 is created over a smaller grid 1202. The height of the larger grid 1201 can be taller than the smaller grid 1202. The smaller grids 1202 can be embedded into the larger grids 1201. The grid lines of the larger grid can protrude from the surface. The larger grids and the smaller grids can be square or rectangular shapes. FIG. 12B illustrates an enlarged single smaller grid of FIG. 12A. Each square of the grid 1203 created by the grid lines of the smaller grid 1202 can protrude from the surface. The dimensions of the patterns can be of any suitable height (or depth), width, and spacing suitable for encouraging, enhancing, and/or promoting endothelization. In the smaller grids 1202, the depth of the protruding grids can range from about 10 nm to about 50 μm; up to about 40 μm; up to about 30 μm; and up to about 20 μm. while the width of the grids from about 10 nm to about 50 μm; up to about 40 μm; up to about 30 μm; and up to about 20 μm; is suitable for cellular ingrowth. The spacing between the grids can vary to allow cellular growth and/or allow for flexibility of the underlying material. In various instances, the larger grid can be created via direct laser writing or interference lithography, while the smaller pattern is created via interference lithography. Direct laser writing can create patterns on even and/or uneven surfaces. Interference lithography may be able to create patterns on even surfaces. While FIGS. 12A and 12B illustrate square shapes of the multidimensional structures, such shapes are merely representative of the overall pattern. As such, multidimensional structures in various implementations can be any geometry to promote, encourage, and/or enhance endothelization, including (but not limited to) circular, ovular, oblong, triangular, quadrilateral (e.g., square, rectangular, rhomboidal, trapezoidal), hexagonal, octagonal, any other regular or irregular shape or polygon, and combinations thereof.

Various methodologies can be utilized to attach an outer skirt and/or inner cover to prosthetic frame (e.g., valvular frame). As discussed herein, in some instances, the skirt and/or cover is synthesized via an additive process. In some instances, the skirt and/or cover is synthesized via an additive process directly onto the frame. In some instances, the skirt and/or cover is synthesized via an additive process directly onto a substrate or textile and then attached to a frame. In some instances, the skirt and/or frame is fabricated via weaving, knitting, and/or braiding and then attached to a frame. In some instances, a woven, knitted, and/or braided textile is attached to a frame by an additive process. In some instances, a woven, knitted, and/or braided textile is attached to a frame by lamination.

Provided in FIG. 13A is an example of a method of attaching a woven, knitted, and/or braided textile to a frame to yield a cover on a prosthetic frame. The prosthetic can be any prosthetic, such as (for example) a valvular prosthetic for heart valve replacement. When the prosthetic is a valvular prosthetic, the cover to be attached can be an outer skirt, an inner cover, or both an outer skirt and an inner cover.

Method 1300 can begin by providing a prosthetic frame. The frame can be a metallic or polymeric material, especially a biocompatible material. Metallic materials that can be used for frames include (but are not limited to) nitinol and stainless steel. Polymeric materials that can be used for frames can be bioresorbable or non-resorbable and include (but are not limited to) Resolvable polymers that can be utilized include (but are not limited to) silk fibroin, chitosan, polycaprolactone (PCL), poly(lactic-co-glycolic acid) (PGLA), poly (β-hydroxybutyrate-co-β-hydroxy valerate) (PHBV), and polyhydroxy butyrate (PHB). Nonresolvable polymers that can be utilized include (but are not limited to) polyolefins, polyethylene terephthalate (PET), thermoplastic polyurethane (TPU), expanded polytetrafluoroethylene (ePTFE), polyvinylpyrrolidone (PVP), and suitable combinations thereof. The frame can be surfaced cleaned and treated. In some instances, the frame is plasma treated to yield functional groups onto the frame that may help attachment of an outer skirt and/or inner cover.

The frame can be coated (1301) with a tie layer (or primer layer). The tie layer can be applied by any coating method, including (but not limited to) spray coating, dip coating, and lamination. In some instances, the tie layer is ultrasonic sprayed onto the frame. Materials that can be utilized for coating the tie layer onto the frame include (but are not limited to) polyolefins, polyethylene terephthalate (PET), thermoplastic polyurethane (TPU), fluorinated ethylene propylene (FEP), siloxanes, and acrylics.

Method 1300 also attaches (1303) a textile to the prosthetic frame onto the tie layer. The textile can be planar or tubular. In some instances, a textile is attached on the outer frame wall to yield an outer skirt. In some instances, a textile is attached on the inner frame wall to yield an inner cover. In some instances, a textile is attached on the outer frame wall to yield an outer skirt and on the inner frame wall to yield an inner cover. When an outer skirt and luminal cover are attached, in some instances a single textile is utilized and that is folded over one edge or both edges of the frame. In some instances, two or more textiles are attached to frame to yield an outer skirt and inner cover.

Generally, the textile should be conformable to the shape of the frame such that the textile can attach to the frame such that the textile is taut and flush. When a planar textile is utilized, a seam can be formed by suturing, laminating, sealing via spraying, or otherwise connecting two opposite ends of the textile together to form a tubular shape. In some embodiments, the two opposite ends abut or overlap such that no gaps are present.

Various textiles can be attached to the frame, including (but not limited) to textiles that are woven, knitted, and/or braided. In some instances, the textile has a low profile. In some instances, the textile has been fabricated as described or similar to as described in Method 500 of FIG. 5.

The textile to be attached to the frame can comprise a low profile. In some instances, the textile for one of an outer cover or an inner cover has a lesser profile than the other. In some instances, the textile for an outer cover has a lesser profile than the textile for an inner cover. In some instances, the textile for an inner cover has a lesser profile than the textile for an outer cover.

In some implementations, the textile for an outer cover comprises a woven textile with about 30 PPI to about 60 PPI and about 30 EPI to about 60 EPI, and the textile for an inner cover comprises a woven textile with about 60 PPI to about 100 PPI and about 60 EPI to about 100 EPI. In some implementations, the textile for an outer cover comprises a woven textile with about 40 PPI and about 40 EPI, and the textile for an inner cover comprises a woven textile with about 80 PPI and about 80 EPI. In some implementations, the textile for an outer cover comprises a woven textile with about 60 PPI to about 100 PPI and about 60 EPI to about 100 EPI, and the textile for an inner cover comprises a woven textile with about 30 PPI to about 60 PPI and about 30 EPI to about 60 EPI. In some implementations, the textile for an outer cover comprises a woven textile with about 80 PPI and about 80 EPI, and the textile for an inner cover comprises a woven textile with about 40 PPI and about 40 PPI.

In some implementations, the textile for an outer cover and the textile for an inner cover are each a knitted textile of about 10 CPI to about 50 CPI and 20 WPI to about 60 WPI, and the textile for the outer cover has a lesser profile than the textile for the inner cover. In some implementations, the textile for an outer cover and the textile for an inner cover are each a knitted textile of about 10 CPI to about 50 CPI and 20 WPI to about 60 WPI, and the textile for the inner cover has a lesser profile than the textile for the outer cover.

In some implementations, the textile is attached such that the textile yarn is of about 45-degrees (±15-degrees), or about −45-degrees (±15-degrees), relative to the tubular axis or valve axis (i.e., the intended direction of blood flow). This orientation can improve stretchability and help in the crimping process of the prosthetic when used within a transcatheter system, decreasing the overall profile of the prosthetic during delivery. To assist in alignment and orientation of a textile onto a frame, one or more fixtures can be provided on the frame. Fixtures can comprise any means for aligning and/or orienting a textile. Examples of fixtures include (but are not limited to) an extruding strut, an extruding tab, an indentation, a crevice, a pin, etc. Fixtures can reduce further processing of a cover after attachment (e.g., prevent the need for laser cutting of cover laminated onto frame).

Various methods can be utilized to attach a textile to the prosthetic frame, such as (for example) sutures, adhesives, lamination, spray coating, or a combination thereof. In some instances, the textile is precoated with a polymer and then laminated onto the frame. In some instances, ultrasonic spray coating is utilized to seal the textile to the frame. Materials that can be utilized for attaching a textile onto the frame via lamination and/or ultrasonic spraying include (but are not limited to) polyolefins, polyethylene terephthalate (PET), thermoplastic polyurethane (TPU), siloxanes, acrylics, and combinations thereof. When multiple polymers are utilized (e.g., for sealing a planar textile to form a tubular shape), the various polymers should have a similar glass transition temperature (Tg).

Method 1300 also optionally compresses (1305) the textile attached to the frame. To compress the textile, a compression tool with a three-dimensional compliant mold can be utilized. The compliant mold can be shaped to fit any type of prosthetic (e.g., a valvular prosthetic). The frame with attached textile can be inserted on and within the compliant mold. A heat-press platen that is conforms to the prosthetic shape can be compressed onto the frame and textile to apply heat and pressure onto the textile and valve. In some instances, a heat-press platen is configured and shaped to contact and compress the outer cover. In some instances, a heat-press platen is configured and shaped to contact and compress an inner cover, including tubular prosthetics. In some instances, a heat-press platen for an outer cover and a heat-press platen for an inner cover are simultaneously utilized to contact and compress the attached textiles. In some instances, a heat-press platen for an outer cover and/or a heat-press platen for an inner cover are utilized with a stationary non-heated base. In some instances, the stationary non-heated base includes a pliable material such as (for example) a cushion foam.

Method 1300 also optionally trims (1305) excess textile material. Trimming can be performed prior to or after attachment of the textile to the frame. Any textile material that extends beyond a frame end or is otherwise not desired can be removed. Any appropriate method to trim the textile can be utilized, such as (for example) die cutting and laser cutting.

When the outer skirt and/or inner cover is composed of multiple materials (e.g., textile with additive layers), laser cutting is precisely controlled to reduce ill effects such as over-molten polymer, blobs of molten polymer, particulates, or discoloring of the polymer. To ensure high quality ends, the laser speed and/or laser energy is controlled.

To mitigate delamination and improve overall durability of the cover, the edge of the cover at one or more ends is cut just beyond a strut or other structural feature of the frame. Provided in FIGS. 13B and 13C is an example of tubular prosthetic frame 1351 with an internal cover 1353 in which the internal cover is cut just beyond the set of struts 1355 of the prosthetic frame. The amount of overhang can vary and is dependent on various parameters such as cover thickness, material, strut size, etc. In some implementations, a length of the overhang is between about 0.1 mm and about 1.0 mm, which can ensure adequate coverage of the frame when the valve is crimped down and the textile stretches to accommodate the fore-lengthening of structure. In some implementations, the overhang has a width that encircles a tubular frame. In some implementations, the length of the overhang is uniform (or substantially uniform) the entire width. In various implementations, the amount of overhang is at or about 0.1 mm, the amount of overhang is or about 0.2 mm, the amount of overhang is or about 0.3 mm, the amount of overhang is or about 0.4 mm, the amount of overhang is or about 0.5 mm, the amount of overhang is or about 0.6 mm, the amount of overhang is or about 0.7 mm, the amount of overhang is or about 0.8 mm, the amount of overhang is or about 0.9 mm, the amount of overhang is or about 1.0 mm. In various implementations, the amount of overhang is between about 0.05 mm and 0.25 mm, the amount of overhang is between about 0.15 mm and 0.35 mm, the amount of overhang is between about 0.25 mm and 0.45 mm, the amount of overhang is between about 0.35 mm and 0.55 mm, the amount of overhang is between about 0.45 mm and 0.65 mm, the amount of overhang is between about 0.55 mm and 0.75 mm, the amount of overhang is between about 0.65 mm and 0.85 mm, the amount of overhang is between about 0.75 mm and 0.95 mm, or the amount of overhang is between about 0.85 mm and 1.05 mm. In regards to cover overhang, “about” is meant to be ±0.05 mm. In some implementations, the cover with overhang lacks sutures for attachment to the frame. In some implementations, the cover comprises a laminated layer or a coated layer, which may be used in attachment of the textile to the frame.

A frame with an outer cover and/or inner cover can be further processed and treated, such as the processes and treatments described herein. Various processes and treatments that can be performed include (but are not limited to) synthesizing additional cover layers, plasma treatment, texturization, coating with biological materials, attachment of leaflets, sterilization, compression into a compressed delivery state, insertion within a delivery catheter, or otherwise preparation for storage.

While specific examples of a method to attach a textile to a frame is described above, one of ordinary skill in the art can appreciate that various steps of the method can be performed in different orders and that certain steps may be optional according to various implementations. As such, it should be clear that the various steps of the method could be used as appropriate to the requirements of specific applications. Furthermore, any of a variety of methods attach a textile to a frame appropriate to the requirements of a given application can be utilized in various implementations.

### DATA EXAMPLES

### Example 1: Electrospinning of Silk Fibroin Fibers

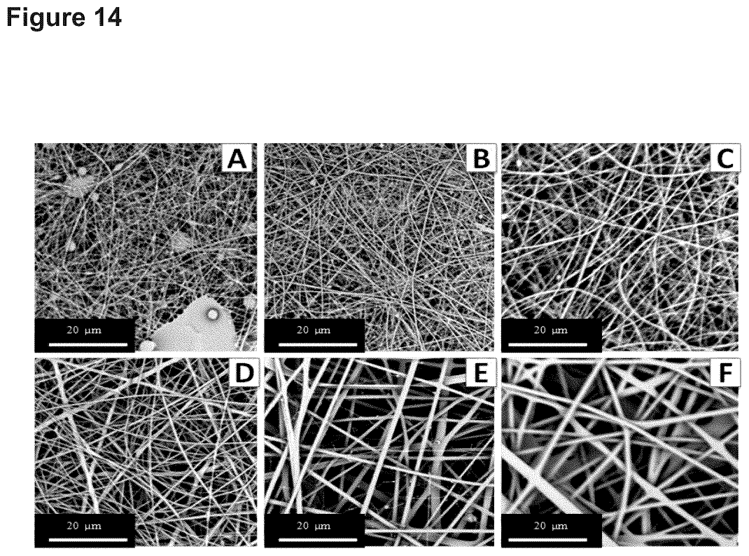

Silk fibroin was electrospun onto a substrate at various concentrations of polymer: 5%, 6%, 7%, 8%, 9% and 10%. SEM images of the fibers were captured (FIG. 14). Concentrations of 5% and 6% yield fibers with some beading. Concentrations of 7%, 8%, 9%, and 10% generated relatively bead-less straight fibers with varying diameters. These results demonstrate the ability to control various properties such as density and porosity.

### Example 2: Fabric Synthesis

A fabric having a low profile was fabricated utilizing PET yarn thread consisting of 18 filaments., the PET yarn thread was warped, drawn, and weaved into a density of 80 epi and 80 ppi. The weaved fabric was scoured with 70% isopropanol and heat pressed at 180° C. for ten minutes, followed by calendaring, resulting in a thickness of about 20-40 μm. The fabric was cut at a 45-degree angle to yield clean edges.

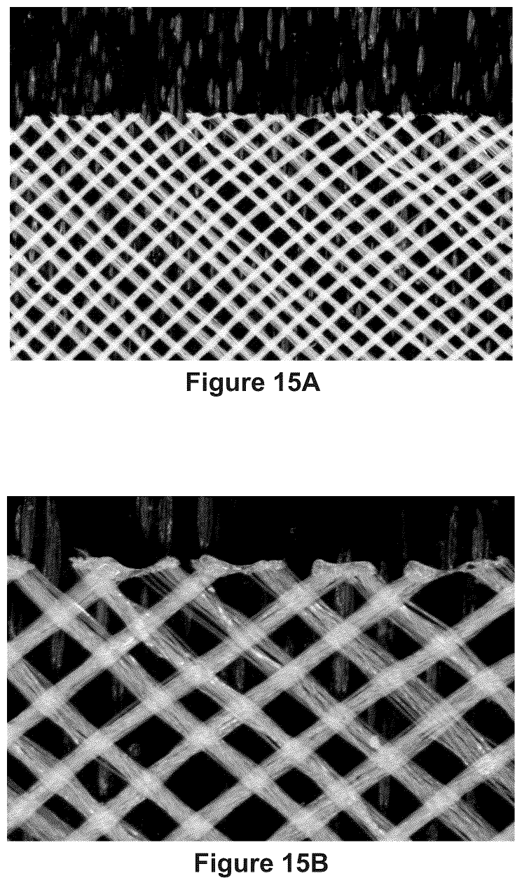

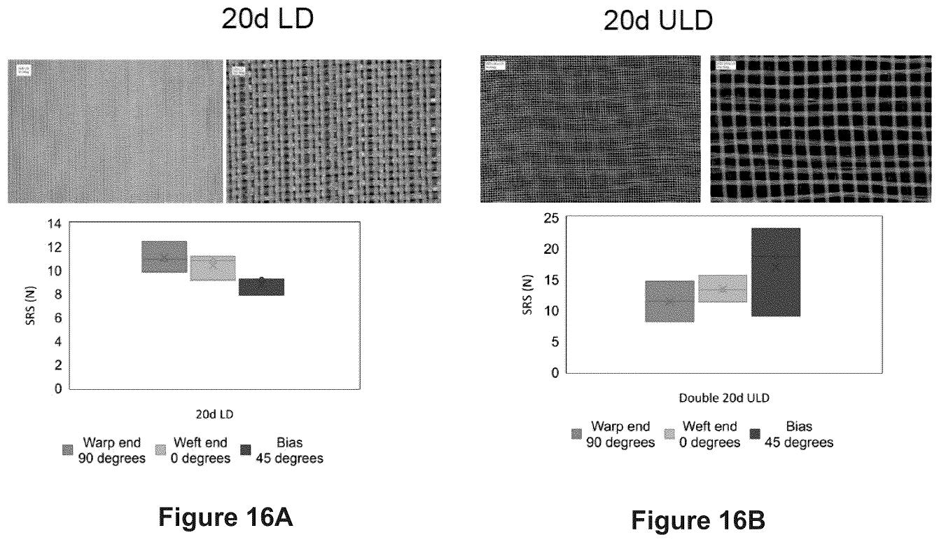

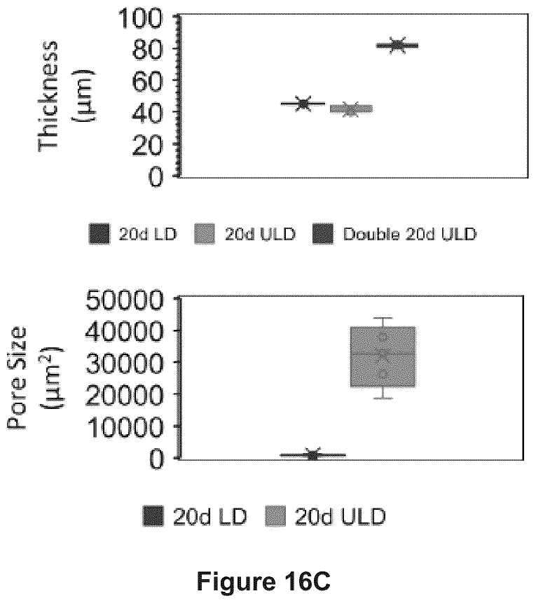

Provided in FIGS. 15A and 15B are the results of the generated textile at 10× (FIGS. 15A) and 30× (FIG. 15B). The textile was compared with a textile having higher density, as shown in FIGS. 16A to 16C.

### Examples

Example 1. A prosthetic heart valve, comprising:
an expandable frame having an outer wall that is covered with an outer skirt and an inner wall that is covered with an inner cover, the outer skirt and the inner cover each composed of electrospun fibers; and
a set of leaflets in connection with the inner cover within the expandable frame.

Example 2. The prosthetic heart valve of example 1, wherein the outer skirt and the inner cover are a single deposit of electrospun fibers, and wherein the single deposit spans and is attached to at least a portion of the outer wall of the expandable frame to form the outer skirt and the single deposit folds across an edge of the expandable frame and spans and is attached to at least a portion of the inner wall of the expandable frame to form the inner cover.

Example 3. The prosthetic heart valve of example 2, wherein the single deposit folds across at least one edge of the expandable frame.

Example 4. The prosthetic heart valve of any one of examples 1-3, wherein the outer skirt or the inner cover is composed of electrospun fibers that comprise at least one of the following resorbable materials: silk fibroin, chitosan, polycaprolactone (PCL), poly(lactic-co-glycolic acid) (PGLA), poly (β-hydroxybutyrate-co-β-hydroxy valerate) (PHBV), and polyhydroxy butyrate (PHB).

Example 5. The prosthetic heart valve of any one of examples 1-4, wherein the outer skirt or the inner cover is composed of electrospun fibers that comprise at least one of the following non-resorbable materials: polyolefins, polyethylene terephthalate (PET), thermoplastic polyurethane (TPU), expanded polytetrafluoroethylene (ePTFE), polyvinylpyrrolidone (PVP), and siloxanes.

Example 6. The prosthetic heart valve of any one of examples 1-5, wherein the outer skirt has greater porosity than the inner cover.

Example 7. The prosthetic heart valve of any one of examples 1-6, wherein the outer skirt comprises more layers of electrospun fibers than the inner cover.

Example 8. The prosthetic heart valve of any one of examples 1-7, wherein the outer skirt has a greater profile than the inner cover.

Example 9. The prosthetic heart valve of example 8, wherein the inner cover is a compressed cover to reduce its profile relative to the outer skirt as well relative to an uncompressed cover.

Example 10. The prosthetic heart valve of any one of examples 1-9, wherein the inner cover comprises a biocompatible material that resists pannus overgrowth.

Example 11. The prosthetic heart valve of any one of examples 1-10, wherein the inner cover comprises fluoropolymers.

Example 12. The prosthetic heart valve of any one of examples 1-11, wherein the inner cover comprises polymers that have undergone controlled chain scission.

Example 13. The prosthetic heart valve of any one of examples 1-12, wherein the inner cover has an even surface topography.

Example 14. The prosthetic heart valve of any one of examples 1-13, wherein the electrospun fibers of the inner cover have been coated with TPU, the TPU coat sealing pores of the electrospun fiber layer.

Example 15. The prosthetic heart valve of any one of examples 1-14, wherein the outer skirt is coated with an endothelialization-inducing biologic.

Example 16. The prosthetic heart valve of any one of examples 1-15, wherein the outer skirt surface comprises pattern features.

Example 17. The prosthetic heart valve of any one of examples 1-16, wherein the outer skirt comprises a gauze-like material.

Example 18. The prosthetic heart valve of any one of examples 1-17, wherein the outer skirt has been plasma treated with O2.

Example 19. The prosthetic heart valve of any one of examples 1-18, wherein the inner cover has been plasma treated with CF4.

Example 20. The prosthetic heart valve of any one of examples 1-19, wherein the electrospun fibers of the outer skirt or of the inner cover comprises crosslinked electrospun fibers.

Example 21. a method of synthesizing a cover for a prosthetic heart valve, comprising:
fitting an expandable frame onto a mandrel, wherein the mandrel comprises or is covered with a polarized material; and
releasing a polymeric solution in a controlled manner in a direction toward the mandrel to generate electrospun fibers that are deposited on the expandable frame to yield an outer skirt spanning at least a portion an outer wall of the expandable frame.

Example 22. The method of example 21, further comprising:
releasing the polymeric solution in the controlled manner in the direction toward the mandrel to generate electrospun fibers that are deposited onto the mandrel adjacent to the expandable frame, wherein the electrospun fibers that are deposited on the expandable frame and the electrospun fibers that are deposited onto the mandrel adjacent to the expandable frame are deposited in a manner to yield a single deposit;
removing the expandable frame and the deposited electrospun fibers from the mandrel; and
folding the portion of the electrospun fibers that were deposited onto the mandrel adjacent to the expandable frame over an edge of the expandable edge to yield an inner cover spanning at least a portion of an inner wall of the expandable frame.

Example 23. The method of example 22, wherein the distance between from where a polymer solution is released and collecting material, solvent concentration, polymer concentration, and the speed of the mandrel are controlled to control thickness of the electrospun fibers or porosity among the electrospun fibers.

Example 24. The method of example 23, wherein the electrospun polymers are deposited such that the outer skirt has greater porosity than the inner cover.

Example 25. The method of example 23 or 24, wherein the electrospun polymers are deposited such that the outer skirt has a greater profile than the inner cover.

Example 26. The method of any one of examples 23-25, wherein viscosity of the polymer solution is controlled by a percentage of polymer in solution to prevent beading.

Example 27. The method of any one of examples 22-26, further comprising:
treating the inner cover with CF4 plasma.

Example 28. The method of any one of examples 21-27, further comprising:
treating the outer skirt with O2 plasma.

Example 29. The method of any one of examples 21-28, further comprising:
prior to the deposition of electrospun fibers onto the frame, treating the frame with plasma.

Example 30. The method of any one of examples 21-29, further comprising:
crosslinking the deposited electrospun fibers.

Example 31. The method of any one of examples 21-30, further comprising:
performing a heat treatment on the deposited electrospun fibers.

Example 32. The method of any one of examples 21-31, further comprising:
performing a freeze-and-thaw treatment on the deposited electrospun fibers.

Example 33. The method of any one of examples 21-32, further comprising:
adhering a porous textile layer onto the outer skirt.

Example 34. The method of any one of examples 22-33, further comprising:
coating the inner cover with TPU.

Example 35. The method of example 34, wherein the TPU coating seals pores of the inner cover.

Example 36. The method of any one of examples 22-35, further comprising:
smoothening the inner cover.

Example 37. The method of any one of examples 21-36, further comprising:
texturizing, embossing, or micropatterning the outer skirt.

Example 38. The method of any one of examples 21 to 37, further comprising:
coating the outer skirt with an endothelialization-inducing biologic.

Example 39. The method of any one of examples 21-38, wherein the electrospun fibers comprise at least one of the following resorbable materials: silk fibroin, chitosan, polycaprolactone (PCL), poly(lactic-co-glycolic acid) (PGLA), poly (β-hydroxybutyrate-co-β-hydroxy valerate) (PHBV), and polyhydroxy butyrate (PHB).

Example 40. The method of any one of examples 21-38, wherein the electrospun fibers that comprise at least one of the following non-resorbable materials: polyolefins, polyethylene terephthalate (PET), thermoplastic polyurethane (TPU), expanded polytetrafluoroethylene (ePTFE), polyvinylpyrrolidone (PVP), and siloxanes.

Example 41. A prosthetic for delivery via a minimally invasive procedure, comprising:
a frame and one or more low-profile covers attached to the frame, wherein each low-profile cover comprises a textile and a polymeric layer, wherein the textile comprises multifilament yarns and is attached to the frame via the polymeric layer without sutures.

Example 42. The prosthetic of example 41, wherein the frame is for use as: a catheter, a heart valve replacement implant, a heart valve repair implant, a vascular °Cclusion device, a vascular stent, or a vascular graft.

Example 43. The prosthetic of example 41 or 42, wherein the textile of at least one low-profile cover is a woven textile having 60 to 100 ends per inch and 60 to 100 picks per inch,.

Example 44. The prosthetic of example 41 or 42, wherein the textile of at least one low-profile cover is a woven textile having 30 to 60 ends per inch and 30 to 60 picks per inch.

Example 45. The prosthetic of example 43 or 44, wherein the woven textile comprises a plain weave, a twill weave, a satin weave, a leno weave, a derivatives of one of the listed weaves, or a combination of the listed weaves.

Example 46. The prosthetic of example 41 or 42, wherein the textile of at least one low-profile cover is a knitted textile having 10 to 50 courses per inch and 20 to 60 wales per inch.

Example 47. The prosthetic of example 41 or 42, wherein the textile of at least one low-profile cover is a braided textile having 30 to 120 picks per inch.

Example 48. The prosthetic of any one of examples 41 to 47, wherein the polymeric layer of at least one low-profile cover comprises a material selected from: polyolefins, polyethylene terephthalate (PET), thermoplastic polyurethane (TPU), expanded polytetrafluoroethylene (ePTFE), siloxanes, and acrylics.

Example 49. The prosthetic of example 48, wherein the polymeric layer comprises a polymer combining thermoplastic polyurethane and polysiloxane.

Example 50. The prosthetic of example 48, wherein the at least one low-profile cover has a profile less than 80 μm thick.

Example 51. The prosthetic of example 50, wherein the at least one low-profile cover has a profile less than 40 μm thick.

Example 52. The prosthetic of example 48, wherein the polymeric layer is a laminated or an ultrasonic coated layer.

Example 53. The prosthetic of any one of examples 41 to 54, wherein the multifilament yarns of at least one low-profile cover are composed of: PET, UHMWPE, PTFE, TPU, or a combination thereof.

Example 54. The prosthetic of any one of examples 41 to 54, the multifilament yarns of at least one low-profile cover are composed of high tenacity PET having a density of greater than 6 g/denier.

Example 55. The prosthetic of example of any one of examples 41 to 57, wherein the multifilament yarns of at least one low-profile cover are positioned about 45-degrees relative a longitudinal axis and/or a latitudinal axis of the prosthetic.

Example 56. The prosthetic of example of any one of examples 41 to 58, wherein an edge of at least one low-profile cover extends beyond a structural feature to yield an overhang.

Example 57. The prosthetic of example of example 56, wherein a length of the overhang is between about 0.1 mm and about 1.0 mm.

Example 58. The prosthetic of example of example 56, wherein the prosthetic is tubular, wherein a width of the overhang encircles the prosthetic.

Example 59. The prosthetic of example of any one of examples 41-56, wherein the prosthetic is tubular, wherein at least one low-profile cover is an outer cover or an inner cover.

Example 60. The prosthetic of example 59, wherein the one or more low-profile covers comprises at least two low-profile covers, a first low-profile cover is an outer cover and second low-profile cover is an inner cover.

Example 61. The prosthetic of example 60, wherein a profile thickness of the outer cover is lesser than a profile thickness of the inner cover, or a profile thickness of the inner cover is lesser than a profile thickness of the outer cover.

Example 62. The prosthetic of example 61, wherein the textile of outer cover and the textile of the inner cover are each woven, wherein the textile of outer cover or the textile of the inner cover is about 30 PPI to about 60 PPI and about 30 EPI to about 60 EPI, and the textile of outer cover or the textile of the inner cover is about 60 PPI to about 100 PPI and about 60 EPI to about 100 EPI.

Example 63. The prosthetic of example 61, wherein the textile of outer cover and the textile of the inner cover are the same knitted textile, wherein the knitted textile is folded over one end of the tubular prosthetic.

Example 64. The prosthetic of example 61 or 63, wherein the textile of outer cover and the textile of the inner cover are each knitted, wherein the textile of outer cover and the textile of the inner cover are each about 10 CPI to about 50 CPI and 20 WPI to about 60 WPI.

Example 65. The prosthetic of example 61, wherein the textile of outer cover and the textile of the inner cover are the same braided textile, wherein the braided textile is folded over one end of the tubular prosthetic.

Example 66. The prosthetic of any one of examples 59 to 66, wherein the inner cover has an even surface topography.

Example 67. The prosthetic of any one of examples 59 to 67, wherein the outer cover comprises pattern features.

Example 68. The prosthetic of any one of examples 59 to 68, wherein the outer cover comprises a coating of an endothelialization-inducing biologic.

Example 69. The prosthetic of any one of examples 59 to 69, wherein the prosthetic is a heart valve replacement implant.

Example 70.A method to attach a low-profile cover on a prosthetic device, comprising:
fabricating a low-profile textile;
applying a tie layer to the prosthetic device; and
attaching the low-profile textile to the prosthetic device onto the tie layer.

Example 71. The method of example 70, wherein the step of fabricating a low-profile textile comprises weft knitting or warp knitting the low-profile textile to have a density of 10 to 50 courses per inch or 20 to 60 wales per inch.

Example 72. The method of example 70, wherein the step of fabricating a low-profile textile comprises braiding the low-profile textile to have a density of 10 to 120 ends per inch.

Example 73. The method of example 70, wherein the step of fabricating a low-profile textile comprises weaving the low-profile textile to have a density of 60 to 100 ends per inch and 60 to 100 picks per inch.

Example 74. The method of example 70, wherein the step of fabricating a low-profile textile comprises weaving the low-profile textile to have a density of 30 to 60 ends per inch and 30 to 60 picks per inch.

Example 75. The method of example 73 or 74, wherein a loom is used for weaving the low-profile textile, and wherein the ends per inch density of the low-profile textile is equal to the reed dents per inch of the loom.

Example 76. The method of any one of examples 70-75, wherein the low-profile textile comprises multifilament yarns having 2 to 160 filaments and are between 5 and 40 denier.

Example 77. The method of example 76, wherein the multifilament yarns are composed of: PET, UHMWPE, PTFE, TPU, or a combination thereof.

Example 78. The method of example 77, wherein the multifilament yarn is composed of high tenacity PET having a density of greater than 6 g/denier.

Example 79. The method of any one of examples 70-78, wherein the step of applying a tie layer comprises spray coating, dip coating, and laminating a material onto the prosthetic device, wherein the material is selected from: a polyolefin, polyethylene terephthalate (PET), thermoplastic polyurethane (TPU), fluorinated ethylene propylene (FEP), a siloxane, or an acrylic.

Example 80. The method of any one of examples 70-79, wherein the prosthetic is tubular.

Example 81. The method of example 80, wherein the step of applying a tie layer comprises applying a tie layer to an inner surface and to an outer surface of the prosthetic.

Example 82. The method of example 81, wherein the step of attaching a low-profile textile to the frame comprises attaching one or more low-profile textiles to yield an inner cover on the inner surface of the prosthetic and an outer cover on the outer surface of the prosthetic.

Example 83. The method of example 82, wherein the step of attaching a low-profile textile to the frame comprises attaching a single low-profile textile to the frame to yield the inner cover and the outer cover, wherein the step of attaching the low-profile textile to the prosthetic further comprises folding the textile over one end of the tubular prosthetic.

Example 84. The method of example 82 or 83, wherein the step of attaching the low-profile textile to the prosthetic further comprises orienting the frame such that the textile yarn is of about 45-degrees (±15-degrees), or about −45-degrees (±15-degrees), relative to the tubular axis.

Example 85. The method of any one of examples 70 to 83, wherein the step of attaching the low-profile textile to the prosthetic further comprises laminating or spray coating a polymer layer onto the low-profile fabric while positioned on the prosthetic.

Example 86. The method of any one of examples 70 to 84 further comprising:
compressing the low-profile textile on the frame.

Example 87. The method of any one of example 70 to 85 further comprising:
trimming off excess material of the low-profile textile.

Example 88. The method of example 87, wherein the step of trimming off excess material of the low-profile textile results in the textile comprising an overhang that extends beyond a structural feature of the prosthetic.

Example 89. The method of example 88, wherein the overhang has length between about 0.1 mm and about 1.0 mm.

Example 90. The method of example 89, wherein the prosthetic is tubular and the overhang has a width that encircles the prosthetic.

Example 91. The method of any one of examples 70 to 89 further comprising:
smoothening a surface of the low-profile textile.

Example 92. The method of any one of examples 70 to 90 further comprising:
texturizing, embossing or micropatterning a surface of the textile.

Example 93. The method of any one of examples 70 to 91 further comprising:
coating a surface of the low-profile textile with biological materials.

Example 94. The method of any one of examples 70 to 92, wherein the prosthetic is a heart valve replacement implant.

Example 95. The method of example 94 further comprising:
attaching a set of leaflets to the prosthetic.

What is claimed is:

1. A prosthetic for delivery via a minimally invasive procedure, comprising:
a frame and one or more low-profile covers attached to the frame, wherein each low-profile cover comprises a textile and a polymeric layer, wherein the textile comprises multifilament yarns and is attached to the frame via the polymeric layer without sutures.

2. The prosthetic of claim 1, wherein the frame is for use as: a catheter, a heart valve replacement implant, a heart valve repair implant, a vascular occlusion device, a vascular stent, or a vascular graft.

3. The prosthetic of claim 1, wherein the textile of at least one low-profile cover is a woven textile having 30 to 100 ends per inch and 30 to 100 picks per inch.

4. The prosthetic of claim 1, wherein the textile of at least one low-profile cover is a woven textile having 30 to 60 ends per inch and 30 to 60 picks per inch.

5. The prosthetic of claim 1, wherein the textile of at least one low-profile cover is a knitted textile having 10 to 50 courses per inch and 20 to 60 wales per inch.

6. The prosthetic of claim 1, wherein the textile of at least one low-profile cover is a braided textile having 30 to 120 picks per inch.

7. The prosthetic of claim 1, wherein the polymeric layer of at least one low-profile cover comprises a material selected from: polyolefins, polyethylene terephthalate (PET), thermoplastic polyurethane (TPU), expanded polytetrafluoroethylene (ePTFE), siloxanes, and acrylics.

8. The prosthetic of claim 7, wherein the polymeric layer comprises a polymer combining thermoplastic polyurethane and polysiloxane.

9. The prosthetic of claim 7, wherein at least one of the low-profile cover has a profile less than 80 μm thick.

10. The prosthetic of claim 7, wherein the at least one low-profile cover has a profile less than 40 μm thick.

11. The prosthetic of claim 7, wherein the polymeric layer is a laminated or an ultrasonic coated layer.

12. The prosthetic of claim 1, the multifilament yarns of at least one low-profile cover are composed of high tenacity PET having a density of greater than 6 g/denier.

13. The prosthetic of claim 1, wherein the multifilament yarns of at least one low-profile cover are positioned about 45-degrees relative a longitudinal axis and/or a latitudinal axis of the prosthetic.

14. The prosthetic of claim 1, wherein an edge of at least one low-profile cover extends beyond a structural feature to yield an overhang.

15. The prosthetic of claim 1, wherein the prosthetic is tubular, wherein at least one low-profile cover is an outer cover or an inner cover.

16. The prosthetic of claim 15, wherein the one or more low-profile covers comprises at least two low-profile covers, a first low-profile cover is an outer cover and second low-profile cover is an inner cover.

17. The prosthetic of claim 16, wherein a singular uncut textile is utilized as the textile for the inner cover and the outer cover, wherein the textile is folded over an end of the prosthetic.

18. The prosthetic of claim 16, wherein a profile thickness of the outer cover is lesser than a profile thickness of the inner cover, or a profile thickness of the inner cover is lesser than a profile thickness of the outer cover.

19. The prosthetic of claim 18, wherein the textile of outer cover and the textile of the inner cover are each woven, wherein the textile of outer cover or the textile of the inner cover is about 30 PPI to about 60 PPI and about 30 EPI to about 60 EPI, and the textile of outer cover or the textile of the inner cover is about 60 PPI to about 100 PPI and about 60 EPI to about 100 EPI.

20. The prosthetic of claim 18, wherein the textile of outer cover and the textile of the inner cover are each knitted, wherein the textile of outer cover and the textile of the inner cover are each about 10 CPI to about 50 CPI and 20 WPI to about 60 WPI.
